(19) United States
(12) Patent Application Publication
(10) Pub. No.: US 2022/0023450 A1
(43) Pub. Date: Jan. 27, 2022

(54) BONE MARROW-, RETICULOENDOTHELIAL SYSTEM-, AND/OR LYMPH NODE-TARGETED RADIOLABELED LIPOSOMES AND METHODS OF THEIR DIAGNOSTIC AND THERAPEUTIC USE

(71) Applicant: Memorial Sloan Kettering Cancer Center, New York, NY (US)

(72) Inventors: Naga Vara Kishore Pillarsetty, Jackson Heights, NY (US); Steven M. Larson, New York, NY (US); Sang-gyu Lee, New York, NY (US)

(21) Appl. No.: 17/274,952

(22) PCT Filed: Sep. 10, 2019
(86) PCT No.: PCT/US19/50490

§ 371 Date: Mar. 10, 2021

Related U.S. Application Data
(60) Provisional application No. 62/729,723, filed on Sep. 11, 2018.

(51) Int. Cl. A61K 51/12; A61K 9/127; A61K 31/355; A61K 51/04; A61K 47/24

(52) U.S. Cl. CPC A61K 51/1234; A61K 9/127; A61K 47/24; A61K 51/0482; A61K 31/355

(57) ABSTRACT

Described herein are compositions comprising liposome-based nanocarriers and associated drugs that selectively target bone marrow, minimize tumor delivery, and maintain high drug concentrations in bone marrow when compared to conventional systemic delivery. The compositions also selectively target lymph nodes and other reticuloendothelial system organs (e.g., spleen, e.g., liver), while minimizing delivery to the tumor in order to deliver drugs that prevent bone marrow suppression (BMS) or aid recovery post exposure to radiation. There are a wide range of scenarios for which such radiation protection is useful, e.g., protection from radiation delivered as part of cancer therapy, radiation from weapons, radiation from materials at a nuclear power plant or nuclear waste site, natural radiation in outer space (e.g., for astronauts), and the like. The described compositions are stable for prolonged periods of time, in some cases over a year in a kit formulation.

### CROSS-REFERENCE TO RELATED APPLICATIONS

This application claims the benefit of U.S. Application No. 62/729,723 filed on Sep. 11, 2018. The subject matter of this application is related, in some respects, to that of International Application No. PCT/US17/21092 filed on Mar. 7, 2017, which claims the benefit of U.S. Application Ser. No. 62/304,814 filed on Mar. 7, 2016, the disclosures of which are hereby incorporated by reference in their entireties.

### GOVERNMENT SUPPORT

This invention was made with government support under grant numbers CA086438 and CA008748 awarded by National Institutes of Health (NIH). The government has certain rights in the invention.

### FIELD OF THE INVENTION

This invention relates generally to radiolabeled liposomes for image guided drug delivery to target tissue, e.g., bone marrow, the reticuloendothelial system, and/or lymph nodes. In particular embodiments, the invention relates to PET-traceable liposomal nanocarriers for imaging tissue, e.g., bone marrow, lymph nodes, and other organs of the reticuloendothelial system and preferential delivery of drugs/agents to target tissue in healthy or tumor bearing animals (e.g., humans).

### BACKGROUND

The reticuloendothelial system is a network of cells and tissues found in the body, for example, in the spleen, liver, lungs, bone marrow, and lymph nodes. The reticuloendothelial cells of the spleen possess the ability to dispose of disintegrated erythrocytes. The reticuloendothelial cells located in the blood cavities of the liver are called Kupffer cells. The Kupffer cells, together with the cells of the general connective tissue and bone marrow, are capable of transforming hemoglobin released by disintegrated erythrocytes into bile pigments.

Bone marrow is a soft, cellular component found inside bones and comprises hematopoietic and stromal stem cells. Bone marrow is also an adipocyte-rich and highly-vascularized environment within bones. Bone marrow components are responsible for several important functions including: maintenance of hematopoiesis, immune balance, bone integrity, and energy metabolism. Accordingly, bone marrow plays a critical role in processes that contribute to the continued survival and maintenance of the body.

Radiation therapy (RT) is a type of treatment for cancer that uses ionizing radiation for killing malignant cells. It is estimated that about 50% of cancer patients undergo RT as part of a treatment regimen at one point during the course of their treatment. The total radiation dose (about 30-80 Gy) is fractionated into multiple small doses and is delivered to the patient over a period of days to weeks. Though the precision of targeting tumor tissue volume for RT has significantly improved with new techniques such as image guided radiation therapy (IGRT) and intensity modulate radiation therapy (IMRT), nearby tissues including bones are unintentionally exposed to radiation. Though RT can cause complete tumor ablation, the debilitating acute and long term side effects of radiation resulting due to spillover of the radiation to nearby organs, including bone and its marrow components, limit the treatment intensity and thereby its efficacy.

Although there are arguments about whether or not there is a threshold for radiation damage to human bone marrow, in populations receiving radiation doses above 50-100 cGy whole body exposure, there is no doubt that there are both direct effects, such as suppression of sensitive cellular blood elements (BMS), and also stochastic effects, such as a statistical increase in myelodysplastic sydrome, leukemia, and solid tumors. These changes are likely mediated by damage and mis-repair of DNA in radiosensitive bone marrow tissue by free radical produced by radiation. Radiation levels capable of producing objective tissue damage of this kind are likely to occur with targeted radiotherapies used in oncology, and also in space travel, especially over long distances or prolonged stays in space. Currently, there are no preventive treatments for these types of damage.

The dynamic and proliferative nature of bone marrow makes it highly sensitive to chemotherapeutic agents, radiation, and other drugs. Unfortunately, a common side effect of treating cancer patients with drugs or radiation is bone marrow depletion or bone marrow suppression (BMS). BMS results from destruction of resident and circulating hematopoietic stem cells (HSCs) and differentiated daughter cells, including radiation sensitive lymphocytes. This can lead to potentially lethal complications such as anemia, neutropenia, and thrombocytopenia. BMS can cause treatment delays, additional hospitalizations, and severely limit aggressive interventions that can potentially eradicate the disease. In addition, radiation toxicity (or hematopoietic syndrome) includes enhanced and prolonged susceptibility to infection and excessive bleeding because of the depletion of circulating white blood cells, platelets, and hematopoietic stem cells in the spleen and bone marrow. Gamma-tocotrienol (GT3) (IUPAC name: (2R)-2,7,8-trimethyl-2-[(3E,7E)-4,8,12-trimethyltrideca-3,7,11-trienyl]-3,4-dihydrochromen-6-ol) shows promise as radioprotectant (RP) but has poor bioavailability after parenteral administration.

Human exposure to whole body (WB) radiation events in the sub-clinical realm will likely increase over the next few decades, due to powerful medical and societal trends including: (1) Expansion of cancer drugs, including internally administered radiotheranostics for otherwise incurable tumors (e.g., metastatic neuroendocrine tumors); Lutetium-177 or Lutathera; Iodine-131 re-induction therapy for thyroid cancer or 1-131 Omburtimab for pediatric tumors; and Radium-223, or Xofigo, for prostate cancer; and (2) Expansion of human space travel, including commercial space aviation (e.g., Virgin Atlantic, SpaceX). A major challenge in using radioprotective agents in radiation therapy settings is the unintentional delivery of the radioprotectant to the tumor. This can result in decreased overall efficacy of the treatment protocol and reduced effect in abrogating the tumor. To this end, selective and adequate drug delivery via systemic administration to human bone marrow has been shown to stimulate specific marrow elements optimally and provide radioprotection during radiotherapy. For example, the use of radiation sensitizers that are selective for cancer cells or agents to protect non-cancer cells from radiation damage termed as radioprotectants have been implemented as potential strategies to overcome complicating issues. However, these methods require drugs to be administered in high concentrations in order to deliver pharmacologically relevant doses to marrow. Further, because there is often a short time window of optimal pharmacologic effects, dosing must be frequently repeated. Unfortunately, frequent and/or high concentrations of dosing often also increases drug delivery to the non-target tissues and can lead to unfavorable outcomes.

Moreover, protection from radiation exposure from nuclear accidents, terrorist acts, and nuclear warfare is considered to be a matter critical to national security, and drugs with some demonstrated effectiveness in humans as radiation protectors or mitigators of high dose whole body exposure are stored in the Strategic National Stockpile (SNS), in the event of a radiologic emergency. So far, only 3 drugs are considered sufficiently active to be stored in the SNS. These drugs are Neulasta, Neupogen GCSF and GMCSF. Unfortunately, these drugs provide only limited protection and improved drugs for more nearly optimal radiation protection are being actively sought.

Therefore, there is an immediate and unmet need to develop agents or modalities that have potential to protect the marrow, stem cell population, and protect vascular niche in the bone from radiation exposure.

### SUMMARY

Described herein are compositions comprising liposome-based nanocarriers and associated drugs that selectively target bone marrow, minimize tumor delivery, and maintain high drug concentrations in bone marrow when compared to conventional systemic delivery. In certain embodiments, the compositions also selectively target lymph nodes and/or other reticuloendothelial system organs (e.g., spleen, e.g., liver), while minimizing delivery to the tumor in order to deliver drugs that prevent bone marrow suppression (BMS) or aid recovery post exposure to radiation. There are a wide range of scenarios for which such radiation protection is useful, e.g., protection from radiation delivered as part of cancer therapy, radiation from weapons, radiation from materials at a nuclear power plant or nuclear waste site, natural radiation in outer space (e.g., for astronauts), and the like.

The present disclosure also describes a kit comprising liposome-based nanocarriers and associated drugs that are stable to physiochemical testing over a two year period. The kit may be used for radioprotection or radiation mitigation applications, for example, in methods of treating, monitoring, and/or imaging a subject and/or patient. In certain embodiments, the described kit comprises a composition comprising a liposome-based nanocarrier and an associated drug, said composition being stable at a dose level 15 mg/ml (total lipid concentration). In certain embodiments, the kit can be stored at 4 degrees C., and can be used for protection against lethal radiation (see, e.g., FIGS. 13A and 13B, e.g., at 4 and 9 Gy acute radiation dose respectively in order to begin stability testing). The ability of this technology to be shipped (e.g., via courier or postal service) (e.g., at 4° C.) and stored over a long time period (e.g., at least 2 weeks, at least 1 month, at least 6 months, at least 1 year, at least 2 years,) allows for these compositions to be available, for example, in the event of a national emergency.

In addition, it is presently discovered that selection of a drug to be associated with the liposome-based nanocarriers can serve a variety of purposes, produce unexpected effects, and be tailored for patient-specific needs and outcomes. For example, a drug can be selected to protect bone marrow cells against radiation, mobilize and recruit stem cells to the bone mafrrow, and stimulate production of erythropoietin in the treatment of myeloid metaplasia.

In certain embodiments, the associated drug comprises a member selected from the group consisting of: a free radical scavenger (e.g., gamma-tocotrienol (GT3); L-ascorbic acid; balsalazide disodium; bilirubin; N-tert-butyl-α-phenylnitrone; caffeic acid; β-carotene; (−)-catechin gallate; DL-α-lipoic acid; ellagic acid; (−)-epicatechin; eugeno; EUK-8; trans-ferulic acid; formononetin; (−)-gallocatechin; ginkgolide B; glutathione; hesperidin; 3-hydroxytyrosol; kaempferol; linoleic acid; luteolin; lycopene; L-lysine; MCI-186; myeloperoxidase inhibitor-I, II, and III; oleic acid; resveratrol; rutin hydrate; seleno-L-methionine; Se-(methyl)selenocysteine hydrochloride; sodium selenite; (±)-taxifolin hydrate; TEMPOL (4-hydroxy-2,2,6,6-tetramethylpiperidine-1-oxyl); (+)-α-tocophero; and xanthophyll); a radioprotectant or radiation mitigator (e.g., Amifostine; N-acetylcystein; alpha-tocotrienol; gamma-tocotrienol; delta-tocotrienol; Genistein; rapamycin; hydroxytryptamine; 60-fullerenol; resveratrol (3,5,4′-trihydroxy-trans-stilbene); human apurinic/apyrimidinic endonuclease (HAP1/APE1); sodium orthovanadate (Na₃VO₄); pifithrin-α (imino-tetrahydrobenzothiazol-tolylethanone hydrobromide); infliximab); a growth factor (e.g., erythropoietin; granulocyte colony-stimulating factor; becaplermin; transforming growth factor beta (TGFβ); platelet-derived growth factor (PDGF); hepatocyte growth factor (HGF); haemopoietin growth factors; telbermin (rhVEGF); FGF-P peptide; FGF1:FGF2 chimeric GF; velafermin (rhFGF-20)); RNAi therapeutics (e.g., GTI-2040 (ribonucleotide reductase); SPC2996 (Bcl-2); LY2181308 (survivin); e.g., immunosuppressors (e.g., Tacrolimus; mTOR inhibitors; corticosteroids; antibiotics; epinephrine analogs; RNAi against Bim and PUMA); cobalt chloride; deferoxamine; clioquinol; isofluran; okadaic acid; tilorone; FG-4497; cerium oxide; butin; antisense-PUMA; inhibitors of GSK-3β; HPV16 E5 viral protein; angiotensin receptor blockers; flagellin analogues; RTA401; autophagy modulators; tenovil (rhIL-10); dekavil (IL-10/F8 fusion); reboxetine; edelfosine); tolcizumab (e.g., anti-IL6 Ab); IL-6 blockers (e.g., A-285222; baicalein); Anakinra (e.g., IL-1 receptor antagonist); anti-CD54 Ab; pravastatin; VEGF blockers; TNP-470; HIF blockers (e.g., PX-478; YC-1); TGFβ blockers (e.g., naringenin; halogunginon; relaxin; SB-525334; SB203580; pirferidone; pentoxyfylline); PDGFR inhibitors (e.g., imatinib; SU9518); retinoic acid; Anti-bFGF; ACE inhibitors; COX inhibitors; Mdm2 inhibitors (e.g., until-1; MI-219); oblimersen sodium (e.g., anti-bcl-2); vanillin derivatives; avotermin (e.g., TGFβ); NF-κB inducers; macrophage activation suppressors; Gap junction inhibitors (e.g., lindane; TPA); NOS-inhibitors (e.g., L-NAME); macrophage activation inhibitors; demethylation targeting agents); a bisphosphonate (e.g., alendronate; ibandronate; risedronic acid; zoledonic acid); a selective estrogen receptor modulator (e.g., raloxifene); a parathyroid hormone modulator (e.g., teriparatide); a biological (e.g., denosumab); a chemotherapeutic drug (e.g., any of the chemotherapeutic drugs listed in Table 1); a CXCR2 agonist (e.g., GROb; GROa) (e.g., for mobilizing cells); a CXCR4 antagonist (e.g., AMD3100; BIO8020) (e.g., for mobilizing stem cells); a HMG-CoA reductase (also known as 3-hydroxy-3-methyl-glutaryl-coenzyme A reductase or HMGCR) inhibitor (e.g., a statin (e.g. simvastatin; atorvastatin; lovastatin; pitavastatin)) for protecting marrow cells); a superoxide dismutase (SOD) mimetic or other mimetics (e.g., manganese(III) 5,10,15,20-tetrakis(4-benzoic acid)porphyrin (MnTBAP); manganese (III) mesotetrakis (N-ethylpyridinium-2-yl) porphyrin (MnTE-2-PyP(5+)) (AOEL 10113)) (e.g., for removing free radicals); an androstenediol (e.g., for stimulating white blood cells (WBCs) and platelet growth); an androgen receptor (AR) agonist (e.g., dihydrotestosterone (DHT); a nonsteroidal selective androgen receptor (SARM) (e.g., BMS-564,929; VK5211) (e.g., for stimulating production of erythropoietin in the treatment myeloid metaplasia; e.g., for use as a radiation syndrome mitigator); and any derivative, equivalent, and/or combination thereof.

In one aspect, the invention is directed to a method of treating, monitoring, and/or imaging a subject, the method comprising: receiving a kit (e.g., via mail, e.g., via courier), the kit comprising a first container (e.g., an ampule, a vial, a cartridge, a reservoir, a lyo-ject, or a pre-filled syringe), wherein the first container contains a composition, said composition comprising (i) a lipid; (ii) an organic polymer; and (iii) gamma-tocotrienol (GT3) (e.g., and/or an equivalent or derivative of GT3), wherein the liposome-based nanocarrier has a surface having a negative charge; storing the kit (e.g., storing the kit at about 4° C.) for a storage duration; and administering the kit to a subject after the storage duration (e.g., wherein the storage duration is at least 2 weeks (e.g., at least 1 month, at least 3 months, at least 6 months, at least 1 year, at least 2 years, at least 3 years)].

In another aspect, the invention is directed to a composition comprising: a liposome-based nanocarrier comprising: (i) a lipid; (ii) an organic polymer; and (iii) gamma-tocotrienol (GT3) (e.g., and/or an equivalent or derivative of GT3), wherein the liposome-based nanocarrier has a surface having a negative charge. In certain embodiments, the liposome-based nanocarrier further comprises a member (e.g., said member encapsulated inside of the liposome-based nanocarrier, incorporated into layers of the lipid, or covalently and/or non-covalently attached to the lipid on the surface of liposome-based nanocarrier) selected from the group consisting of: a radioprotectant or radiation mitigator (e.g., Amifostine, N-acetylcystein, alpha-tocotrienol, gamma-tocotrienol, delta-tocotrienol, Genistein, rapamycin); a growth factor (e.g., erythropoietin, granulocyte colony-stimulating factor, RNAi therapeutics (e.g., GTI-2040 (ribonucleotide reductase), SPC2996 (Bcl-2), LY2181308 (survivin), e.g., immunosuppressors (e.g., Tacrolimus, mTOR inhibitors, corticosteroids, antibiotics, epinephrine analogs, RNAi against Bim and PUMA)); a bisphosphonate (e.g., alendronate, ibandronate, risedronic acid, zoledonic acid); a selective estrogen receptor modulator (e.g., raloxifene); a parathyroid hormone modulator (e.g., teriparatide); a biological (e.g., denosumab); a chemotherapeutic drug (e.g., any of the chemotherapeutic drugs listed in Table 1); a CXCR2 agonist (e.g., GROb, GROa) (e.g., for mobilizing stem cells); a CXCR4 antagonist (e.g., AMD3100, BIO8020) (e.g., for mobilizing stem cells); a HMG-CoA reductase (also known as 3-hydroxy-3-methyl-glutaryl-coenzyme A reductase or HMGCR) inhibitor (e.g., a statin (e.g. simvastatin, atorvastatin, lovastatin, pitavastatin)) for protecting marrow cells;

a superoxide dismutase (SOD) mimetic or other mimetics (e.g., manganese(III) 5,10,15,20-tetrakis(4-benzoic acid)porphyrin (MnTBAP), manganese (III) mesotetrakis (N-ethylpyridinium-2-yl) porphyrin (MnTE-2-PyP(5+)) (AOEL 10113)) (e.g., for removing free radicals); an androstenediol (e.g., for stimulating white blood cells (WBCs) and platelet growth); and an androgen receptor (AR) agonist (e.g., dihydrotestosterone (DHT), a nonsteroidal selective androgen receptor (SARM) (e.g., BMS-564,929, VK5211) (e.g., for stimulating production of erythropoietin in the treatment of myeloid metaplasia).

In certain embodiments, the nanocarrier has up to 30 mol % GT3 (e.g., up to 24 mol % GT3, e.g., up to 10 mol % GT3) of the total moles comprising the nanocarrier.

In certain embodiments, the lipid comprises one or more members selected from the group consisting of: cholesterol, 1,2-dioleoyl-sn-glycero-3-phosphoethanolamine-N-(succinyl) (succinyl-PE), 1,2-distearoyl-sn-glycero-3-phosphocholine (DSPC), cholesterol, 1,2-distearoyl-sn-glycero-3-phosphoethanolamine (DSPE), 1,2-dipalmitoyl-sn-glycero-3-phosphoethanolamine-N-(succinyl) (succinyl-DPPE), and 1,2-distear In certain embodiments, oyl-sn-glycero-3-phosphoethanolamine-N-[methoxy(polyethylene glycol)] (mPEG-DSPE). In certain embodiments, mPEG-DSPE is 1,2-distearoyl-sn-glycero-3-phosphoethanolamine-N-[methoxy(polyethylene glycol)-2000] (mPEG2000-DSPE).

In certain embodiments, the lipid comprises cholesterol, DSPC, mPEG-DSPE, and succinyl-DPPE. In certain embodiments, the lipid comprises cholesterol, DSPC, mPEG-DSPE, and succinyl-PE.

In certain embodiments, the lipid is labeled with an isotope. In certain embodiments, the isotope comprises a member selected from the group consisting of ³H, ⁶⁴Cu, ⁶⁶Ga, ⁸⁶Y¹¹¹In, ⁶⁷Ga, ⁶⁸Ga, ⁸⁹Zr, ^{124/131}I, and ¹⁷⁷Lu.

In certain embodiments, the isotope is labeled through binding to a chelator. In certain embodiments, the chelator comprises a member selected from the group consisting of 1,4,7,10-tetraazacyclododecane-1,4,7,10-tetraacetic acid (DOTA), 1,4,7-triazacyclononane-1,4,7-trisacetic acid (NOTA), and 1-(4-isothiocyanatophenyl)-3-[6,17-dihydroxy-7,10,18,21-tetraoxo-27-(N-acetylhydroxylamino)-6,11,17,22-tetraazaheptaeicosine] thiourea (DFO). In certain embodiments, the chelator comprises a member selected from the group consisting of DOTA-Bn-DSPE, NOTA-Bn-DSPE, and DFO-Bz-DSPE.

In certain embodiments, the organic polymer comprises polyethylene glycol (PEG).

In certain embodiments, the nanocarrier comprises 1,2-distearoyl-sn-glycero-3-phosphoethanolamine-N-methoxy(polyethylene glycol) (mPEG-DSPE).

In certain embodiments, the concentration of mPEG-DSPE is from about 0.5 mole % to about 10 mole % of the total moles of the lipid comprising the nanocarrier. In certain embodiments, the concentration of mPEG-DSPE is from 0.5 mol % to about 1.5 mol % of the total moles of the lipid comprising the nanocarrier. In certain embodiments, the concentration of mPEG-DSPE is about 1 mole % of the total moles of the lipid comprising the nanocarrier. In certain embodiments, the concentration of succinyl-DPPE is from 5 mole % to 15 mole % (e.g., about 7 mole % to about 12 mole %, about 10 mole %) of the total moles of the lipid comprising the nanocarrier.

In certain embodiments, the liposome-based nanocarrier is at least 70 mol % lipid (e.g., at least 80 mole % lipid, at least 90 mole % lipid, at least 98 mole % lipid).

In certain embodiments, the liposome-based nanocarrier has an average diameter in a range from 30 nm to 300 nm. In certain embodiments, the liposome-based nanocarrier has an average diameter in a range from about 70 nm to about 110 nm. In certain embodiments, the liposome-based nanocarrier has an average diameter of about 90 nm.

In certain embodiments, the concentration of DPSC is from about 50 mole %-70 mole % (e.g., 55 mole %-75 mole %, e.g., about 60 mole %) of the total moles of the lipid comprising the nanocarrier.

In certain embodiments, the concentration of cholesterol is from about 25 mole %-45 mole % (e.g., about 25 mole %-35 mole %, e.g., about 40 mole %) of the total moles of the lipid comprising the nanocarrier.

In certain embodiments, the concentration of succinyl-PE is about 5 mole % to 15 mole % (e.g., about 7 mole % to 13 mole %, e.g., about 10 mole %) of the total moles of the lipid comprising the nanocarrier.

In certain embodiments, the composition has one or more of (i), (ii), and (iii), as follows: (i) from 3 to 20 mole % succinyl-DPPE; (ii) from 0.5 to 2 mole % mPEG-DSPE; and (iii) from 5 to 26 mole % an associated drug.

In certain embodiments, the negative charge of the surface of the liposome-based nanocarrier as measured via the zeta potential at a pH of about 7.4 has a magnitude from about 5 mV to 25 mV (e.g., about 10 mV to 20 mV).

In certain embodiments, a radiolabeling efficiency of the liposome-based nanocarrier is greater than 60% (e.g., greater than 70%) over at least a period of time after preparation. In certain embodiments, the period of time is at least 2 weeks (e.g., at least 1 month, e.g., at least 3 months, e.g., at least 6 months, e.g., at least 12 months, e.g., at least 24 months).

In certain embodiments, the diameter of the liposome varies no more than 30% (e.g., no more than 20%) over the period of time after preparation. In certain embodiments, the period of time is at least 2 weeks (e.g., at least 1 month, e.g., at least 3 months, e.g., at least 6 months, e.g., at least 12 months, e.g., at least 24 months).

In certain embodiments, the zeta potential as measured at about a pH 7.4 varies over a magnitude of no more than 5 mV (e.g., no more than 2.5 mV).

In another aspect, the invention is directed to a method for imaging a subject the method comprising: administering to the subject any of the compositions described herein, wherein the lipid is labeled with an isotope.

In certain embodiments, the method comprises obtaining and displaying a positron emission tomography (PET), single-photon emission computed tomography (SPECT), and/or Positron emission tomography-computed tomography (PET/CT) image of at least one tissue of the subject comprising the composition.

In certain embodiments, the method comprises quantitatively measuring a distribution of the composition in at least one tissue of the subject. In certain embodiments, the method comprises quantitatively measuring the distribution of the composition in an organ of the reticuloendothelial system. In certain embodiments, the organ comprises a member selected from the group consisting of: liver, spleen, and bone marrow.

In certain embodiments, the method comprises determining a concentration and/or total amount of delivered radiolabeled drug in the tissue based on a positron emission tomography (PET), single-photon emission computed tomography (SPECT), or Positron Emission Tomography-Computed Tomography (PET/CT) image of the tissue.

In certain embodiments, the method comprises quantitatively measuring the distribution of the composition in one or more lymph nodes and/or in bone marrow and/or in spleen.

In certain embodiments, the administered composition demonstrates selective targeting of bone marrow of the subject such that concentration of the composition in bone marrow is at least 3 fold greater than the concentration of the composition in any of the tumor tissue at a given time following administration of the composition, wherein the given time is at least 1 hour following administration.

In certain embodiments, the method comprises capturing and displaying a sequence of PET images in real time.

In another aspect, the invention is directed to a method of treating a subject, the method comprising administering any one of the compositions described herein to the subject suffering from or susceptible to a disease and/or condition.

In certain embodiments, the disease and/or condition comprises a member selected from the group consisting of bone marrow suppression (BMS), myelodysplastic syndrome (MDS), acute myeloid leukemia (AML), chronic myeloid leukemia (CML), sepsis, graft-versus-host-disease (GVHD), bone metastasis, osteoporosis, and myeloid metaplasia. In certain embodiments, the disease and/or condition comprises exposure to radiation.

In certain embodiments, the method comprises, after administering the composition, administering a chemotherapeutic and/or radiation therapy.

In certain embodiments, the method comprises, before administering the composition at one or more time points (e.g., six time points), administering a chemotherapeutic and/or radiation therapy.

In certain embodiments, the composition is administered at the dosage from about 15 to about 50 mg/kg (e.g., at about 32 mg/kg, e.g., at about 40 mg/kg).

In certain embodiments, the administered composition demonstrates selective targeting of bone marrow of the subject such that the concentration of the liposome-based nanocarrier in bone marrow is at least 3 fold greater than the concentration of the liposome-based nanocarrier in any of the tumor tissue at a given time following administration of composition, wherein the given time is at least 1 hour, following administration.

In certain embodiments, the administered composition protects bone marrow cells against radiation (e.g., wherein the subject has received or will receive radiation exposure, e.g., from a chemotherapy drug, radiation treatment, and/or other radiation exposure).

In certain embodiments, the administered composition mobilizes and recruits stem cells to the bone marrow (e.g., wherein the subject has received or will receive radiation exposure, e.g., from a chemotherapy drug, radiation treatment, and/or other radiation exposure).

In certain embodiments, the administered composition stimulates production of erythropoietin (e.g., wherein the subject has received or will receive radiation exposure, e.g., from a chemotherapy drug, radiation treatment, and/or other radiation exposure).

In another aspect, the invention is directed to a method of monitoring a patient, the method comprising: administering the composition of any one of claims 2 to 34 to a patient suffering from or susceptible to a disease and/or condition; and investigating a quantity of drug (e.g., a drug currently or having been associated with the liposome-based nanocarrier of the composition) delivered to at least one tissue of the patient.

In another aspect, the invention is directed to a method of imaging an organ of the reticuloendothelial system in a subject, the method comprising: detecting radiation from any of the liposome-based nanocarriers/compositions described herein, the subject having been administered the composition. In certain embodiments, the radiation is detected via an external PET imaging system.

In certain embodiments, the organ comprises a member selected from the group consisting of active bone marrow, liver, and spleen.

In certain embodiments, the method comprises displaying an image corresponding to the detected radiation, the image visually distinguishing active bone marrow from other tissue and, optionally, quantifying the concentration of drug and/or liposome-based nanocarrier.

In another aspect, the invention is directed to a composition comprising: a liposome-based nanocarrier comprising: (i) a lipid; (ii) an organic polymer; and (iii) gamma-tocotrienol (GT3) (e.g., and/or an equivalent or derivative of GT3), and an associated drug (e.g., wherein the associated drug is encapsulated inside of the liposome-based nanocarrier, incorporated into layers of the lipid, or covalently and/or non-covalently attached to the lipid on the surface of liposome-based nanocarrier), for use in a method of treating a disease and/or condition in a subject, wherein the treating comprises delivering the composition to the subject.

In another aspect, the invention is directed to a composition comprising: a liposome-based nanocarrier comprising: (i) a lipid; (ii) an organic polymer; and (iii) gamma-tocotrienol (GT3) (e.g., and/or an equivalent or derivative of GT3), wherein the liposome-based nanocarrier has a surface having a negative charge, and an associated drug (e.g., wherein the associated drug is encapsulated inside of the liposome-based nanocarrier, incorporated into layers of the lipid, or covalently and/or non-covalently attached to the lipid on the surface of liposome-based nanocarrier), for use in a method of monitoring of a disease and/or condition in a subject, wherein the monitoring comprises delivering the composition to the subject.

In another aspect, the invention is directed to a composition comprising: a liposome-based nanocarrier comprising: (i) a lipid; (ii) an organic polymer; and (iii) gamma-tocotrienol (GT3) (e.g., and/or an equivalent or derivative of GT3), wherein the liposome-based nanocarrier has a surface having a negative charge, and an associated drug (e.g., wherein the associated drug is encapsulated inside of the liposome-based nanocarrier, incorporated into layers of the lipid, or covalently and/or non-covalently attached to the lipid on the surface of liposome-based nanocarrier), for use in (a) a method of treating a disease and/or condition in a subject or (b) in a method of monitoring of a disease and/or condition in a subject, wherein the monitoring comprises delivering the composition to the subject.

In another aspect, the invention is directed to a composition comprising: a liposome-based nanocarrier comprising: (i) a lipid; (ii) an organic polymer; and (iii) gamma-tocotrienol (GT3) (e.g., and/or an equivalent or derivative of GT3), wherein the liposome-based nanocarrier has a surface having a negative charge, and an associated drug (e.g., wherein the associated drug is encapsulated inside of the liposome-based nanocarrier, incorporated into layers of the lipid, or covalently and/or non-covalently attached to the lipid on the surface of liposome-based nanocarrier), for use in therapy.

In another aspect, the invention is directed to a kit (e.g., for use in radioprotection or radiation mitigation, e.g., for use in methods of treating, monitoring, and/or imaging a subject and/or patient) comprising: a first container (e.g., an ampule, a vial, a cartridge, a reservoir, a lyo-ject, or a pre-filled syringe), wherein the first container contains any of the compositions described herein.

In certain embodiments, the kit comprises a neutralizing solution, wherein the neutralizing solution is contained in a second container (e.g., wherein the neutralizing solution adjusts the pH to about pH 7.0-7.4) (e.g., wherein the neutralizing solution comprises potassium carbonate, e.g., 500 mM potassium carbonate). In certain embodiments, the neutralizing solution comprises potassium carbonate.

In certain embodiments, the kit comprises a Solution A, wherein the Solution A is contained in a third container. (e.g., wherein the Solution A enhances labeling efficiency, e.g., wherein Solution A reduces pH) (e.g., wherein the Solution A comprises ammonium acetate, e.g., 500 mM ammonium acetate). In certain embodiments, the Solution A comprises ammonium acetate.

In certain embodiments, the kit comprises a radiolabel (e.g., ⁶⁴Cu, e.g., ⁶⁴Cu—Cl₂, e.g., ⁸⁹Zr), wherein the radiolabel is contained in a fourth container.

In another aspect, the invention is directed to a method for preparing a composition (e.g., a composition of any one of claims 1 to 32 or 63 to 67), the method comprising: contacting (e.g., via gentle swirling and/or mixing) a first solution with a substance comprising a radiolabel (e.g., ⁶⁴Cu, e.g., ⁶⁴Cu—Cl₂, e.g., ⁸⁹Zr) to generate a second solution (e.g., wherein the average labeling yield is greater than 70%, greater than 80%, greater than 90%), wherein the first solution comprises a liposome-based nanocarrier comprising: (i) a lipid; (ii) an organic polymer; and (iii) gamma-tocotrienol (GT3) (e.g., and/or an equivalent or derivative of GT3), wherein the liposome-based nanocarrier has a surface having a negative charge; heating (e.g., at 50° C.) the second solution for a period of time (e.g., for about 10 minutes, for about 20 minutes, for about 30 minutes, for about 40 minutes); and contacting (e.g., aseptically) (e.g., via gentle swirling and/or mixing) the second solution with a Neutralizing Solution to generate a third solution (e.g., thereby adjusting pH of the third solution to a value of about 7.0 to about 7.4) (e.g., wherein the third solution comprises the composition of any one of claims 1 to 14 or 37 to 41) (e.g., wherein the third solution has a pH value of from about 7.0 to about 7.4).

In certain embodiments, the method comprises contacting (e.g., aseptically) (e.g., via gentle swirling and/or mixing) a Solution A with the liposome-based nanocarrier, thereby generating the first solution [e.g., enhances efficiency particularly where radiolabel comprises ⁶⁴Cu] wherein the Solution A enhances radiolabeling efficiency, e.g., wherein Solution A reduces pH of the first solution (e.g., wherein the Solution A comprises ammonium acetate, e.g., 500 mM ammonium acetate)].

In certain embodiments, the Neutralizing Solution comprises potassium carbonate, e.g., 500 mM potassium carbonate.

In another aspect, the invention is directed to a method for using any of the kits described herein, the method comprising: receiving the kit (e.g., via mail, e.g., via courier); storing the kit (e.g., storing the kit at about 4° C.) for a storage duration; and administering the kit to a subject after the storage duration [e.g., wherein the storage duration is at least 2 weeks (e.g., at least 1 month, at least 3 months, at least 6 months, at least 1 year, at least 2 years, at least 3 years)].

In certain embodiments, the method comprises transporting the kit (e.g., sending via mail, e.g., sending via courier) at a temperature of about 4° C. (e.g., about 2° C., about 5° C., about 10° C.).

In another aspect, the invention is directed to a composition comprising: a liposome-based nanocarrier comprising: (i) a lipid; (ii) an organic polymer, wherein the liposome-based nanocarrier has a surface having a negative charge; and an associated drug (e.g., wherein the associated drug is encapsulated inside of the liposome-based nanocarrier, incorporated into layers of the lipid, or covalently and/or non-covalently attached to the lipid on the surface of liposome-based nanocarrier).

In certain embodiments, the associated drug comprises a member selected from the group consisting of: a free radical scavenger (e.g., gamma-tocotrienol (GT3)); a radioprotectant or radiation mitigator (e.g., Amifostine, N-acetylcystein, alpha-tocotrienol, gamma-tocotrienol, delta-tocotrienol, Genistein, rapamycin); a growth factor (e.g., erythropoietin, granulocyte colony-stimulating factor, RNAi therapeutics (e.g., GTI-2040 (ribonucleotide reductase), SPC2996 (Bcl-2), LY2181308 (survivin), e.g., immunosuppressors (e.g., Tacrolimus, mTOR inhibitors, corticosteroids, antibiotics, epinephrine analogs, RNAi against Bim and PUMA)); a bisphosphonate (e.g., alendronate, ibandronate, risedronic acid, zoledonic acid); a selective estrogen receptor modulator (e.g., raloxifene);

a parathyroid hormone modulator (e.g., teriparatide); a biological (e.g., denosumab); a chemotherapeutic drug (e.g., any of the chemotherapeutic drugs listed in Table 1); a CXCR2 agonist (e.g., GROb, GROa) (e.g., for mobilizing stem cells); a CXCR4 antagonist (e.g., AMD3100, BIO8020) (e.g., for mobilizing stem cells); a HMG-CoA reductase (also known as 3-hydroxy-3-methyl-glutaryl-coenzyme A reductase or HMGCR) inhibitor (e.g., a statin (e.g. simvastatin, atorvastatin, lovastatin, pitavastatin)) for protecting marrow cells; a superoxide dismutase (SOD) mimetic or other mimetics (e.g., manganese(III) 5,10,15,20-tetrakis(4-benzoic acid)porphyrin (MnTBAP), manganese (III) mesotetrakis (N-ethylpyridinium-2-yl) porphyrin (MnTE-2-PyP(5+)) (AOEL 10113)) (e.g., for removing free radicals); an androstenediol (e.g., for stimulating white blood cells (WBCs) and platelet growth); and an androgen receptor (AR) agonist (e.g., dihydrotestosterone (DHT), a nonsteroidal selective androgen receptor (SARM) (e.g., BMS-564,929, VK5211) (e.g., for stimulating production of erythropoietin in the treatment of myeloid metaplasia).

In certain embodiments, the nanocarrier has up to 30 mole % GT3 (e.g., up to 24 mole % GT3, e.g., up to 10 mole % GT3) of the total moles comprising the nanocarrier.

In certain embodiments, the lipid comprises one or more members selected from the group consisting of: cholesterol; 1,2-dioleoyl-sn-glycero-3-phosphoethanolamine-N-(succinyl) (succinyl-PE); 1,2-distearoyl-sn-glycero-3-phosphocholine (DSPC); 1,2-distearoyl-sn-glycero-3-phosphoethanolamine (DSPE); 1,2-dipalmitoyl-sn-glycero-3-phosphoethanolamine-N-(succinyl) (succinyl-DPPE); and 1,2-distearoyl-sn-glycero-3-phosphoethanolamine-N-[methoxy(polyethylene glycol)] (mPEG-DSPE).

In certain embodiments, mPEG-DSPE is 1,2-distearoyl-sn-glycero-3-phosphoethanolamine-N-[methoxy(polyethylene glycol)-2000] (mPEG2000-DSPE).

In certain embodiments, the lipid comprises cholesterol, DSPC, mPEG-DSPE, and succinyl-PE.

In certain embodiments, the lipid is labeled with an isotope. In certain embodiments, the isotope comprises a member selected from the group consisting of ³H, ⁶⁴Cu, ⁶⁶Ga, ⁸⁶Y, ¹¹¹In, ⁶⁷Ga, ⁶⁸Ga, ⁸⁹Zr, ^{124/131}I, and ¹⁷⁷Lu. In certain embodiments, the isotope is labeled through binding to a chelator. In certain embodiments, the chelator comprises a member selected from the group consisting of 1,4,7,10-tetraazacyclododecane-1,4,7,10-tetraacetic acid (DOTA), 1,4,7-triazacyclononane-1,4,7-trisacetic acid (NOTA), and 1-(4-isothiocyanatophenyl)-3-[6,17-dihydroxy-7,10,18,21-tetraoxo-27-(N-acetylhydroxylamino)-6,11,17,22-tetraazaheptaeicosine] thiourea (DFO). In certain embodiments, the chelator comprises a member selected from the group consisting of DOTA-Bn-DSPE, NOTA-Bn-DSPE, and DFO-Bz-DSPE.

In certain embodiments, the organic polymer comprises polyethylene glycol (PEG).

In certain embodiments, the nanocarrier comprises 1,2-distearoyl-sn-glycero-3-phosphoethanolamine-N-methoxy(polyethylene glycol) (mPEG-DSPE).

In certain embodiments, the concentration of mPEG-DSPE is from about 0.5 mole % to about 10 mole % of the total moles of the lipid comprising the nanocarrier. In certain embodiments, the concentration of mPEG-DSPE is from 0.5 mole % to about 1.5 mole % of the total moles of the lipid comprising the nanocarrier. In certain embodiments, the concentration of mPEG-DSPE is about 1 mole % of the total moles of the lipid comprising the nanocarrier.

In certain embodiments, the concentration of succinyl-DPPE is from 5 mole % to 15 mole % (e.g., about 7 mole % to about 12 mole %, about 10 mole %) of the total moles of the lipid comprising the nanocarrier.

In certain embodiments, the liposome-based nanocarrier is at least 70 mole % lipid (e.g., at least 80 mole % lipid, at least 90 mole % lipid, at least 98 mole % lipid).

In certain embodiments, the liposome-based nanocarrier has an average diameter in a range from 30 nm to 300 nm (e.g., from about 70 nm to about 110 nm, e.g., about 90 nm).

In certain embodiments, the concentration of DPSC is from about 50 mole %-70 mole % (e.g., 55 mole %-75 mole %, e.g., about 60 mole %) of the total moles of the lipid comprising the nanocarrier.

In certain embodiments, the concentration of cholesterol is from about 25 mole %-45 mole % (e.g., about 25 mole % l-35 mole %, e.g., about 40 mole %) of the total moles of the lipid comprising the nanocarrier.

In certain embodiments, the concentration of succinyl-PE is about 5 mole % to 15 mole % (e.g., about 7 mole % to 13 mole %, e.g., about 10 mole %) of the total moles of the lipid comprising the nanocarrier.

In certain embodiments, the negative charge of the surface of the liposome-based nanocarrier as measured via the zeta potential at a pH of about 7.4 has a magnitude from about 5 mV to 25 mV (e.g., about 10 mV to 20 mV).

In certain embodiments, a radiolabeling efficiency of the liposome-based nanocarrier is greater than 60% (e.g., greater than 70%) over at least a period of time after preparation. In certain embodiments, the period of time is at least 2 weeks (e.g., at least 1 month, e.g., at least 3 months, e.g., at least 6 months, e.g., at least 12 months, e.g., at least 24 months).

In certain embodiments, the diameter of the liposome varies no more than 30% (e.g., no more than 20%) over the period of time after preparation. In certain embodiments, the period of time is at least 2 weeks (e.g., at least 1 month, e.g., at least 3 months, e.g., at least 6 months, e.g., at least 12 months, e.g., at least 24 months).

In certain embodiments, the zeta potential as measured at about a pH 7.4 varies over a magnitude of no more than 5 mV (e.g., no more than 2.5 mV).

Elements of embodiments involving one aspect of the invention (e.g., methods) can be applied in embodiments involving other aspects of the invention (e.g., compositions), and vice versa.

### Definitions

In order for the present disclosure to be more readily understood, certain terms are first defined below. Additional definitions for the following terms and other terms are set forth throughout the specification.

In this application, the use of “or” means “and/or” unless stated otherwise. As used in this application, the term “comprise” and variations of the term, such as “comprising” and “comprises,” are not intended to exclude other additives, components, integers or steps. As used in this application, the terms “about” and “approximately” are used as equivalents. Any numerals used in this application with or without about/approximately are meant to cover any normal fluctuations appreciated by one of ordinary skill in the relevant art. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

“Administration”: The term “administration” refers to introducing a substance into a subject. In general, any route of administration may be utilized including, for example, parenteral (e.g., intravenous), oral, topical, subcutaneous, peritoneal, intraarterial, inhalation, vaginal, rectal, nasal, introduction into the cerebrospinal fluid, or instillation into body compartments. In some embodiments, administration is oral. Additionally or alternatively, in some embodiments, administration is parenteral. In some embodiments, administration is intravenous.

“Biocompatible”: The term “biocompatible”, as used herein is intended to describe materials that do not elicit a substantial detrimental response in vivo. In certain embodiments, the materials are “biocompatible” if they are not toxic to cells. In certain embodiments, materials are “biocompatible” if their addition to cells in vitro results in less than or equal to 20% cell death, and/or their administration in vivo does not induce inflammation or other such adverse effects. In certain embodiments, materials are biodegradable.

“Biodegradable”: As used herein, “biodegradable” materials are those that, when introduced into cells, are broken down by cellular machinery (e.g., enzymatic degradation) or by hydrolysis into components that cells can either reuse or dispose of without significant toxic effects on the cells. In certain embodiments, components generated by breakdown of a biodegradable material do not induce inflammation and/or other adverse effects in vivo. In some embodiments, biodegradable materials are enzymatically broken down. Alternatively or additionally, in some embodiments, biodegradable materials are broken down by hydrolysis. In some embodiments, biodegradable polymeric materials break down into their component polymers. In some embodiments, breakdown of biodegradable materials (including, for example, biodegradable polymeric materials) includes hydrolysis of ester bonds. In some embodiments, breakdown of materials (including, for example, biodegradable polymeric materials) includes cleavage of urethane linkages.

“Biomolecule”: As used herein, “biomolecule” refers to bioactive, diagnostic, and prophylactic molecules. Biomolecules that can be used in the present invention include, but are not limited to, synthetic, recombinant or isolated peptides and proteins such as antibodies and antigens, receptor ligands, enzymes, and adhesion peptides; nucleotides and polynucleic acids such as DNA and antisense nucleic acid molecule; activated sugars and polysaccharides; bacteria; viruses; and chemical drugs such as antibiotics, antiinflammatories, and antifungal agents.

“Carrier”: As used herein, “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Suitable pharmaceutical carriers are described in “Remington's Pharmaceutical Sciences” by E. W. Martin.

“Free radical scavenger”: As used herein “free radical scavenger” refers to a drug that removes the ionization induced by radiation in tissues (e.g., human tissues).

“Radiolabel”: As used herein, “radiolabel” refers to a moiety comprising a radioactive isotope of at least one element. Exemplary suitable radiolabels include but are not limited to those described herein. In certain embodiments, a radiolabel is one used in positron emission tomography (PET). In certain embodiments, a radiolabel is one used in single-photon emission computed tomography (SPECT). In certain embodiments, radioisotopes comprise ³H, ^{99m}Tc, ¹¹¹In, ⁶⁴Cu, ⁶⁷Ga, ¹⁸⁶Re, ¹⁸⁸Re, ¹⁵³Sm, ¹⁷⁷Lu, ⁶⁷Cu, ¹²³I, ¹²⁴I, ¹²⁵I, ¹¹C, ¹³N, ¹⁵O, ¹⁸F, ¹⁸⁶Re, ¹⁸⁸Re, ¹⁵³Sm, ¹⁶⁶Ho, ¹⁷⁷Lu, ¹⁴⁹Pm, ⁹⁰Y, ²¹³Bi, ¹⁰³Pd, ¹⁰⁹Pd, ¹⁵⁹Gd, ¹⁴⁰La, ¹⁹⁸Au, ¹⁹⁹Au, ¹⁶⁹Yb, ¹⁷⁵Yb, ¹⁶⁵Dy, ¹⁶⁶Dy, ⁶⁷Cu, ¹⁰⁵Rh, ¹¹¹Ag, ⁸⁹Zr, ²²⁵Ac, ¹³⁷Cs, and ¹⁹²Ir.

“Subject”: As used herein, the term “subject” includes humans and mammals (e.g., mice, rats, pigs, cats, dogs, and horses). In many embodiments, subjects are mammals, particularly primates, especially humans. In some embodiments, subjects are livestock such as cattle, sheep, goats, cows, swine, and the like; poultry such as chickens, ducks, geese, turkeys, and the like; and domesticated animals particularly pets such as dogs and cats. In some embodiments (e.g., particularly in research contexts) subject mammals will be, for example, rodents (e.g., mice, rats, hamsters), rabbits, primates, or swine such as inbred pigs and the like.

“Small molecule”: As used herein, the term “small molecule” can refer to a non-polymeric molecule, for example, or a species less than 5000 Da.

“Therapeutic agent”: As used herein, the phrase “therapeutic agent” refers to any agent that has a therapeutic effect and/or elicits a desired biological and/or pharmacological effect, when administered to a subject.

“Therapeutic index”: As used herein, the phrase “therapeutic index” refers to a concentration in target tissue in relationship to normal tissues in the region. For example, “therapeutic index” refers to a concentration in the bone marrow compared to a concentration in the surrounding soft tissue (e.g., muscle).

“Treatment”: As used herein, the term “treatment” (also “treat” or “treating”) refers to any administration of a substance that partially or completely alleviates, ameliorates, relives, inhibits, delays onset of, reduces severity of, and/or reduces incidence of one or more symptoms, features, and/or causes of a particular disease, disorder, and/or condition. Such treatment may be of a subject who does not exhibit signs of the relevant disease, disorder and/or condition and/or of a subject who exhibits only early signs of the disease, disorder, and/or condition. Alternatively or additionally, such treatment may be of a subject who exhibits one or more established signs of the relevant disease, disorder and/or condition. In some embodiments, treatment may be of a subject who has been diagnosed as suffering from the relevant disease, disorder, and/or condition. In some embodiments, treatment may be of a subject known to have one or more susceptibility factors that are statistically correlated with increased risk of development of the relevant disease, disorder, and/or condition.

Drawings are presented herein for illustration purposes, not for limitation.

### BRIEF DESCRIPTION OF DRAWINGS

The foregoing and other objects, aspects, features, and advantages of the present disclosure will become more apparent and better understood by referring to the following description taken in conduction with the accompanying drawings, in which:

FIG. 1 shows a graph depicting gamma-tocotrienol (GT3) release from BMT liposomes.

FIG. 2 shows a graph depicting biodistribution of ³H labeled GT3 between liposomal formulation and free form dissolved in ethanol (EtOH).

FIG. 3A is a plot of CBC data showing white blood cell (WBC) recovery after total body irradiation (TBI) (4 Gy).

FIG. 3B is a plot of lymphocyte recovery after TBI (4 Gy).

FIG. 4 shows a plot of GT3-Nano radiation protection efficacy in lethally radiated C57/BL6 mice (n=10). 80% Mice that were administered GT3-Nano at 50 mg/kg were rescued from death. All control mice died by day 17.

FIGS. 5A-5C show plots of blood cell recovery after total body irradiation (9 Gy).

FIG. 6A shows a vial containing 99MTc sulfur colloid, which is currently used as the gold standard for imaging bone marrow distribution in humans.

FIG. 6B shows a ⁶⁴Cu BMT Liposomes Kit in accordance with an embodiment of the present disclosure. The present disclosure describes that the compositions of the present disclosure outperform the current standard.

FIGS. 7A-7C show plots representative of the stability of a ⁶⁴Cu BMT Liposomes Kit. The liposome formulation is stable for over two years at 4° C.

FIG. 8 shows a representation of a GT3-loaded liposome “GT3-Nano” in accordance with an embodiment of the present disclosure. GT3-Nano is a non-radioactively labeled formulation that uses the BMT-Lipo platform to encapsulate the radioprotectant GT3. In certain embodiments, the GT3-Nano formulation can be radiolabeled to non-invasively track the distribution of the liposome in the body of a subject.

FIG. 9 shows a graph depicting biodistribution of [⁶⁴Cu] labeled selective targeting liposomes at 24 h post injection. SBMT: Spleen, Bone Marrow Targeting; LNT: Lymph Node Targeting; TT: Tumor Targeting.

FIGS. 10A-10C show a schematic (FIG. 10A) and plots (FIGS. 10B-10C) that depict white blood recovery of [Sm-153]-EDTMP treatment with SBMT-GT3-LIPO. The data is presented as mean±SEM.

FIG. 10A shows a schematic of a [Sm-153]-EDTMP and SBMT-GT3-LIPO injection schedule and blood collection scheme, according to an illustrative embodiment of the present disclosure. 1.5 mCi of Sm-153 was administered to two groups (5 mice per group) of mice via i.v. 40 mg/kg SBMT-GT3-LIPO was administered 24 h prior to [Sm-153]-EDTMP injection and additional 40 mg/kg SBMT-GT3-LIPO were administered at day 1, 4, 8, 11, 15, and 18.

FIG. 10B shows a plot depicting white blood cell (WBC) counts (K/uL) at 4, 7, 14, 21, 28, 42 and 99 days after treatment.

FIG. 10C shows a plot depicting lymphocyte counts (K/uL) at 4, 7, 14, 21, 28, 42 and 99 days after treatment.

FIGS. 11A-11F show a schematic and plots depicting characterization of GT3 incorporated bone marrow targeting liposomes (GT3-Nano).

FIG. 11A shows a structure of a radioactive, PET labeled GT3-Nano.

FIG. 11B shows a graph depicting zeta potentials of BMT liposomes with different GT3 contents. Zeta potential changes with different amount of GT3 incorporation were determined as −14.2±1.3, −14.4±1.7, and −12.2±1.5 at pH 7.4 for 2, 12, and 24% mol GT3 containing liposomes respectively as determined at pH 7.4. Zeta potential of BMT liposome without GT3 is −16.8±1.2 mV. Doxil type liposome, which consists of HSPC, cholesterol, and mPEG-DSPE (3:1:1 by weight) is −2.4±0.7 mV at pH 7.4.

FIG. 11C shows a graph depicting ⁶⁴Cu labeling of GT3 containing BMT liposomes shows 100% labeling of ⁶⁴Cu on iTLC.

FIG. 11D shows a graph depicting GT3 incorporation into liposome. DOTA-Bn-DSPE was synthesized by coupling p-SCN-Bn-DOTA and DSPE and identified by mass spectrum. At 13, 17, 20 and 26 mol % of initial GT3 composition in the lipid mixture, 100% of GT3 can be incorporated into BMT liposome.

FIGS. 11E and 11F each show a plot depicting 6, 10, 15 and 20 mol % of GT3 and 2 μCi [³H]-GT3 were added to lipid mixture of BMT liposomes to form lipid film. After extrusion, purified GT3-BMT-LIPOs using PD-10 column was dialyzed against PBS in 20 k MWCO dialysis cassette. 100 μl of solution in the dialysis cassette was collected to measure 3H activity at 0, 4, 20, 28, 44, 52, 68 and 140 h.

FIGS. 12A and 12B show ex vivo biodistribution data of (FIG. 12A) ⁶⁴Cu labeled SBMT-GT3-Lipo and (FIG. 12B) [³H]-GT3-Nano.

FIGS. 13A-13B show schematics and plots depicting efficacy of SBMT-GT3-LIPO.

FIG. 13A shows a schematic and plots depicting white blood cell recovery after total body irradiation (4Gy) 10 mg/kg/mice. SBMT-GT3-LIPO were treated via i.v. and blood was collected by retro-orbital bleeding on day 0, 1, 4, 7, 14, 21, 43 and 99. GT3 treated groups showed fast lymphocyte recovery. GT3-Lipo treated 24 h before TBI, GT3-Lipo treated 24 h after TBI, BMT only and no treatment.

FIG. 13B shows a schematic and plots depicting dose dependent survival improvement of mice at lethal radiation dose. The left graph shows that C57/BL6 (10 wks) were administered with SBMT-GT3-Lipo (50 mg/kg GT3) irradiation. 9 Gy total body irradiation (¹³⁷Cs, dose rate=89 cGy/min) was given 24 h after GT3 administration, N=10 per group. The right graph shows that CD2F1 mice were administered with SBMT-GT3-Lipo at 16, 24, 32 and 42 mg/kg and BMT-LIPO was given equivalent amount of lipids to 42 mg/kg GT3-Nano. 9.2 Gy total body irradiation (⁶⁰Co, dose rate=10 Gy/min) was given 24 h after SBMT-GT3-Lipo administration, N=16 per group).

FIG. 14A shows a plot depicting HSC cell population changes of bone marrow. After C57BL/6 mice were irradiated at 4 Gy with or without SBMT-GT3-LIPO, bone marrows were collected and analyzed by FACS. Myeloid-biased MPP subsets 2 (MPP2) and common myeloid progenitors (CMP) showed statistically significant recovery than other HSC subpopulations.

FIG. 14B shows images depicting immunohistochemistry a frozen spleen section of a C57BL/6 mouse. 100 μL of NBD-SBMT-LIPO containing 50 μg of NBD was injected 7 weeks old mice. The spleen was collected in OCT media and frozen. The frozen sections were labeled with anti-CD105 and anti-CD31 antibody, NBS-SBMT-LIPO is localized in white pulp region. CD105 is localized higher density in white pulp region but CD105 also found in white pulp region. CD31 is localized in the peripheral region of white pulp and red pulp region. Composite image shows that SBMT-LIPO is colocalized mostly with CD105 there are few regions (arrows) where SBMT-LIPO, CD105 and CD31 are colocalized.

### DETAILED DESCRIPTION

Throughout the description, where compositions are described as having, including, or comprising specific components, or where methods are described as having, including, or comprising specific steps, it is contemplated that, additionally, there are compositions of the present invention that consist essentially of, or consist of, the recited components, and that there are methods according to the present invention that consist essentially of, or consist of, the recited processing steps.

It should be understood that the order of steps or order for performing certain action is immaterial so long as the invention remains operable. Moreover, two or more steps or actions may be conducted simultaneously.

The mention herein of any publication, for example, in the Background section, is not an admission that the publication serves as prior art with respect to any of the claims presented herein. The Background section is presented for purposes of clarity and is not meant as a description of prior art with respect to any claim.

The present disclosure describes compositions comprising a radioprotectant drug capable of shielding and restoring key hematopoietic stem cells and progenitor cells in bone marrow and spleen (e.g., based on selective phagocytosis by cells of the fixed mononuclear phagocytic system (MPS)). The described compositions function as non-toxic radiation protection and mitigation compositions for hematopoietic syndrome at both sub-lethal and lethal levels of whole body (WB) radiation exposure under conditions of both acute and chronic radiation exposure.

The compositions of the present disclosure target the spleen and bone marrow, where the restorative hematopoietic stem cells reside.

In certain embodiments, the compositions comprise particles such as liposomes. When liposomes are introduced into the blood-stream, the liposomes are rapidly cleared from the circulation by phagocytic cells of the innate immune system in liver, spleen, bone marrow, and lymph nodes. Without wishing to be bound to any theory, it was hypothesized that if liposomal particles were loaded with a radiation mitigator and/or protector such as GT3, sinusoidal endothelial cells (as well as other essential stem cell types in spleen and bone marrow that are responsible for restoration of bone marrow function after radiation damage) could be protected.

The present disclosure describes that radiation disrupts a strong link between the hematopoietic cells and phagocytic cells in the bone marrow in both experimental animals (rabbits) and man. For example, at low doses of radiation, the more sensitive hematopoietic cells were markedly suppressed, while the phagocytic cells were relatively unaffected. Over time, the relationship was restored by recovery of hematopoietic cells to baseline levels using the described compositions. At higher doses of radiation, both cell types were suppressed, although the time course of phagocytic cells function decline was much slower. Recovery of hematopoietic cells was restored only if there was complete restoration of the phagocytic cell function.

As described herein, the present disclosure demonstrates efficacy of the described compositions for treatment of acute radiation syndrome in mice. As described herein, fluorescent tags on the compositions revealed that specialized phagocytic cells of the innate immune system, which line the sinusoids of the bone marrow and spleen, are a major site of uptake and retention of these liposomes. The described compositions showed more rapid and complete recovery of white cells in the blood at sub-lethal radiation (4 Gy), which correlated with an increase in specific myeloid progenitor cells in the bone marrow. At high dose radiation (up to 9.2 Gy), a dose response was observed leading to “rescue (survival)” of 80-90% of mice with no detectable long-term effects after 100 days observation period.

In WO201755958A1, the inventors described methods of making the described liposome-based nanocarriers. For example, 1,4,7,10-tetraazacyclododecane-1,4,7,10-tetraacetic acid (ROTA) was used as a chelator (e.g., for ⁶⁴Cu) and distearoyl-phosphatidylethanolamine (DSPE) was used as a lipid to anchor in lipid bilayer. Moreover, the solvent used for reaction was chloroform, methanol, and water, all of which are removed during evaporation to make a lipid film.

The amount of succinyl-DPPE content was adjusted to modulate the surface charge on the liposomes. In certain embodiments, a negative charge on the liposome surface as measured by the zeta potential at a pH of 7.4 was achieved by adding commercially available succinyl-DPPE to a concentration of 10 mole % of the total lipid. Liposome size was controlled by the pore size of extrusion membrane and dynamic light scattering was used to measure the properties of the liposomes. To control liposome size, 100 nm and 30 nm pore membranes were used, generating liposomes of 140 nm and 90 nm. The poly dispersity index (PDI) was between 0.038 and 0.096, which indicates liposomes were formed with a uniform and narrow size distribution.

International Application No. PCT/US17/21092 filed on Mar. 7, 2017, entitled “Bone Marrow-Reticuloendothelial System and/or Lymph Node-targeted Radiolabeled Liposomes and Methods of their Diagnostic and Therapeutic Use,” (published as WO2017155948A1 on Sep. 14, 2017), the contents of which is hereby incorporated by reference in its entirety described a series of compositions comprising liposomal formulations, which were used to evaluate liposomal targeting to tissues within the Monocyte Phagocytic System (MPS) or the Reticuloendothelial system (RES) (e.g., liver, lymph nodes, spleen and bone marrow). Based on liposomal size, surface charge, and pegylation, the inventors found three patterns of in vivo distribution, leading to selective enrichment of nanoparticle uptake in 1) spleen, bone marrow; 2) lymph-nodes; and 3) tumor sites, respectively. Liver uptake was similar in all three liposomal formulations.

Moreover, methods of imaging and mapping the bone marrow and/or other reticuloendothelial system organs using the described liposome-based nanocarriers are described by Inventors Pillarsetty, Larson, and Lee in WO2017155948. These methods provide high resolution non-invasive and quantitative imaging via PET, which offers advantages over conventional imaging/tracking methods.

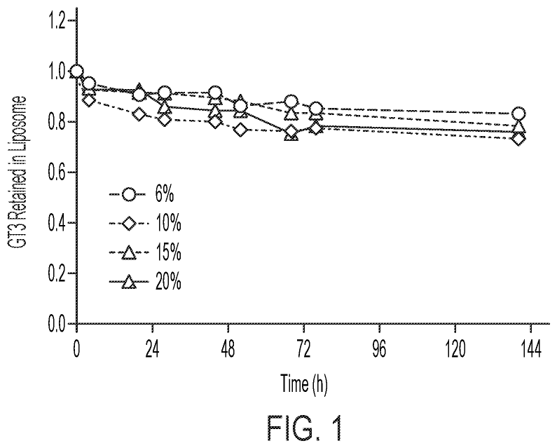

It is presently discovered that selection of a drug to be associated with the liposome-based nanocarriers can serve a variety of purposes, produce unexpected effects, and be tailored for patient-specific needs and outcomes. For example, as shown in FIGS. 1-5, the drug can protect bone marrow cells against radiation, mobilize and recruit stem cells to the bone marrow, and stimulate production of erythropoietin in the treatment of myeloid metaplasia.

The associated drug can be any of the following: a free radical scavenger (e.g., gamma-tocotrienol (GT3)); a radioprotectant or radiation mitigator (e.g., Amifostine, N-acetylcystein, alpha-tocotrienol, gamma-tocotrienol, delta-tocotrienol, Genistein, rapamycin); a growth factor (e.g., erythropoietin, granulocyte colony-stimulating factor, RNAi therapeutics (e.g., GTI-2040 (ribonucleotide reductase), SPC2996 (Bcl-2), LY2181308 (survivin), e.g., immunosuppressors (e.g., Tacrolimus, mTOR inhibitors, corticosteroids, antibiotics, epinephrine analogs, RNAi against Bim and PUMA)); a bisphosphonate (e.g., alendronate, ibandronate, risedronic acid, zoledonic acid); a selective estrogen receptor modulator (e.g., raloxifene); a parathyroid hormone modulator (e.g., teriparatide); a biological (e.g., denosumab); a chemotherapeutic drug (e.g., any of the chemotherapeutic drugs listed in Table 1); a CXCR2 agonist (e.g., GROb, GROa) (e.g., for mobilizing stem cells); a CXCR4 antagonist (e.g., AMD3100, BIO8020) (e.g., for mobilizing stem cells); a HMG-CoA reductase (also known as 3-hydroxy-3-methyl-glutaryl-coenzyme A reductase or HMGCR) inhibitor (e.g., a statin (e.g. simvastatin, atorvastatin, lovastatin, pitavastatin)) for protecting marrow cells); a superoxide dismutase (SOD) mimetic or other mimetics (e.g., manganese(III) 5,10,15,20-tetrakis(4-benzoic acid)porphyrin (MnTBAP), manganese (Ill) mesotetrakis (N-ethyl pyridinium-2-yl) porphyrin (MnTE-2-PyP(5+)) (AOEL 10113)) (e.g., for removing free radicals); an androstenediol (e.g., for stimulating white blood cells (WBCs) and platelet growth); an androgen receptor (AR) agonist (e.g., dihydrotestosterone (DHT), a nonsteroidal selective androgen receptors (SARM) (e.g., BMS-564,929, VK5211)) (e.g., for stimulating production of erythropoietin in the treatment of myeloid metaplasia); and any combination thereof.

In certain embodiments, the drug (e.g., GT3) to be associated with the liposomes is incorporated as a percentage of moles of the total lipid into bone marrow targeting liposomes within a range from e.g., from 1 to 30 mol %, e.g., from 2 to 26 mol %, e.g., e.g., about 2 mol %, e.g., about 12 mol %, e.g., about 24 mol %, e.g., 13 mol %, e.g., 17 mol %, e.g., 20 mol %, e.g., 26 mol %. mol %.

In certain embodiments, the composition has an average diameter in a range from 30 nm to 300 nm. In certain embodiments, the liposome-based nanocarrier has an average diameter in a range from 50 nm to 200 nm. In certain embodiments, the liposome-based nanocarrier has an average diameter in a range from about 60 nm to 150 nm. In certain embodiments, the liposome-based nanocarrier has an average diameter in a range of 75 nm to 105 nm. In certain embodiments, the liposome-based nanocarrier has an average diameter that is about 90 nm. In certain embodiments, the liposome-based nanocarrier has an average diameter that is about 140 nm.

In certain embodiments, the composition has a negative charge, and the negative charge of the surface of the liposome-based nanocarrier has a magnitude from 5 mV to 25 mV as measured using the zeta potential at a pH of about 7.4.

FIG. 1 shows a graph depicting GT3 release from BMT liposomes. The graph shows that 6, 10, 15, and 10 mol % of GT3 and 2 μCi [3H]-GT3 were added to lipid mixture of BMT liposomes to form lipid film. After extrusion, purified GT3-BMT-LIPOs using PD-10 column was dialyzed against PBS in 20,000 MWCO dialysis cassette. 100 mL of solution in the dialysis cassette was collected to measure 3H activity at 0, 4, 20, 28, 44, 52, 68 and 140 h.

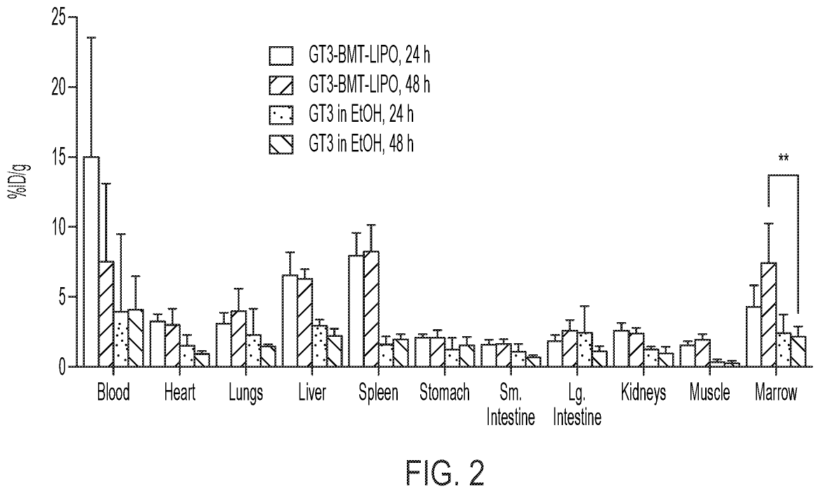

FIG. 2 shows a graph depicting biodistribution of ³H labeled GT3 between liposomal formulation and free form dissolved in EtOH. N=5 per group. C57/BL6 mice were administered 10 mg/kg GT3 either i.v. (liposome) or i.p. (free) and organ was collected. Tissues were dissolved in 500 μl Soluene-350 overnight and 5 mL EcoScint A was added.

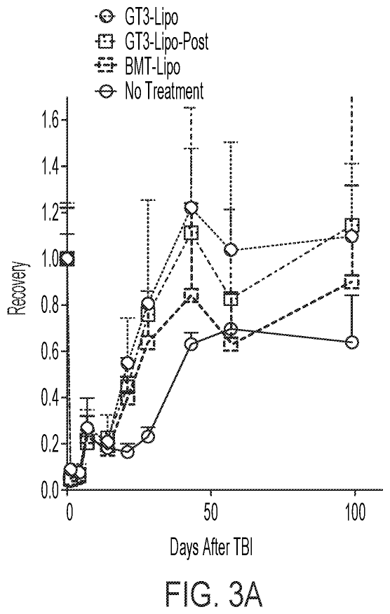

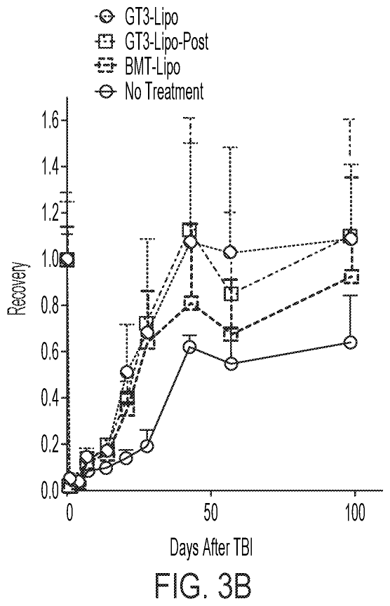

FIGS. 3A and 3B a plot of complete blood count (CBC) data showing white blood cell (WBC) recovery after total body irradiation (TBI) (4 Gy) (FIG. 3A), and a plot of lymphocyte recovery after TBI (4 Gy) (FIG. 3B). C57BL6 mice were divided into 4 groups (5 mice per group). (Filled circle) GT3-Lipo treated 24 h before TBI. (Open square) BMT-Lipo treated 24 h before TBI. (Filled square) GT3-Lipo treated 24 h after TBI. (Open circle) No treatment. 10 mg/kg/mice GT3 were treated via i.v. and blood was collected by retro-orbital bleeding on day 0, 1, 4, 7, 14, 21, 43, 57 and 99.

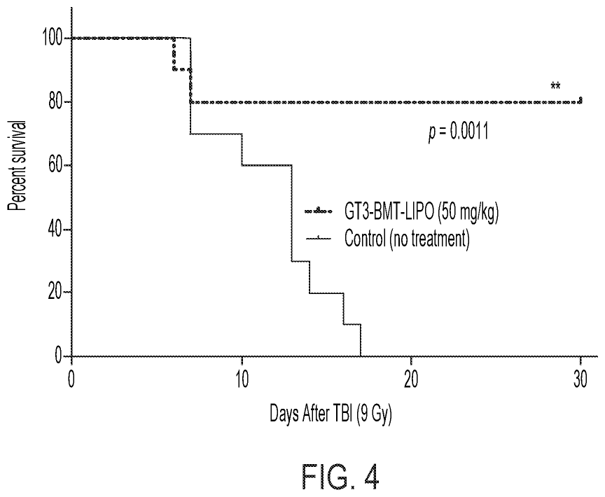

FIG. 4 shows a plot of GT3-Nano radiation protection efficacy in lethally radiated mice (n=10). C57BL/6 mice were treated with GT3-Nano at 50 mg/kg 24 hours prior to 9 Gy radiation from Cesium-137 source. Control mice did not get any treatment. Survival of mice was monitored with no additional supportive care. 80% of mice that were administered GT3-Nano at 50 mg/kg were rescued from death. All control mice died by day 17.

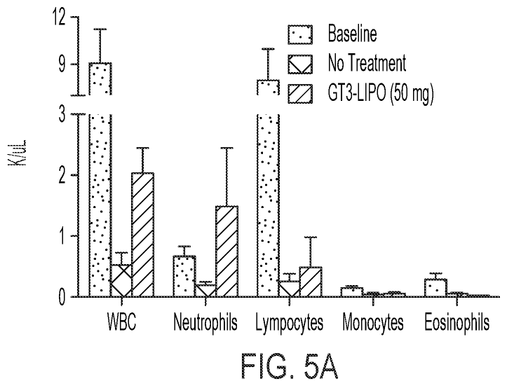

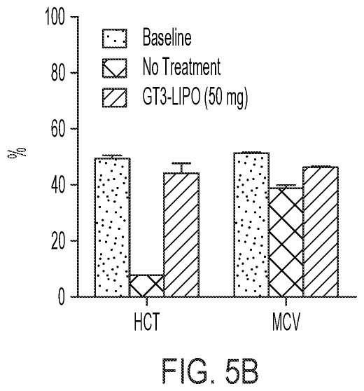

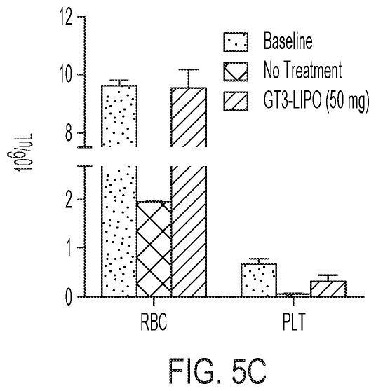

FIGS. 5A-5C show plots of blood cell recovery after total body irradiation (9 Gy). Mice treated at 50 mg/kg GT3-LIPO were treated via i.v. 24 h prior to 9 Gy total body irradiation. During the survival experiment, blood from “no treatment” mice was collected by cardiac puncture after euthanasia on day 17, which was when the mice were determined to be at a terminal disease stage by a veterinarian at RARC. Blood from GT3-LIPO was collected by retro-orbital blood collection on day 17. Baseline data was drawn from prior experimentation.

Table 1 shows chemical structures and IUPAC name of exemplary small molecule drugs that can be used to treat osteoporosis.

TABLE 1

| Drug name | Chemical Structure | IUPAC name |
|---|---|---|
| Alendronate | 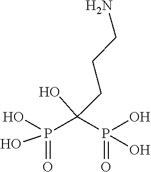 [SMILES: NCCCC(O)(P(=O)(O)O)P(=O)(O)O] | sodium [4-amino-1-hydroxy-1- (hydroxy-oxido-phosphoryl)- butyl]phosphonic acid trihydrate |
| lbandronate | 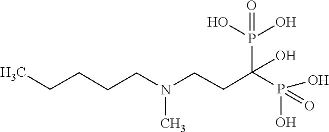 | {1-Hydroxy-3- [methyl(pentyl)amino]propane- 1,1-diyl}bis(phosphonic acid) |
| Risedronic acid | 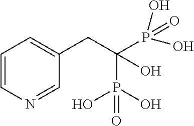 [SMILES: O=P(O)(O)C(O)(Cc1cccnc1)P(=O)(O)O] | (1-hydroxy-1-phosphono-2- pyridin-3-yl-ethyl)phosphonic acid |
| Zoledronic acid | 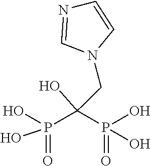 [SMILES: O=P(O)(O)C(O)(Cn1ccnc1)P(=O)(O)O] | [1-hydroxy-2-(1H-imidazol-1- yl)ethane-1,1-diyl]bis(phosphonic acid) |
| Raloxifene | 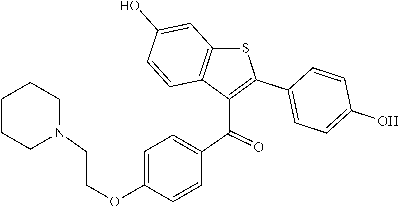 [SMILES: O=C(c1ccc(OCCN2CCCCC2)cc1)c1c(-c2ccc(O)cc2)sc2cc(O)ccc12] | [6-hydroxy-2-(4-hydroxyphenyl)- benzothiophen-3-yl]- [4-[2-(1- piperidyl)ethoxy]phenyl]- methanone |

### “Kit” Formulation: Preparation and Stability Experiments

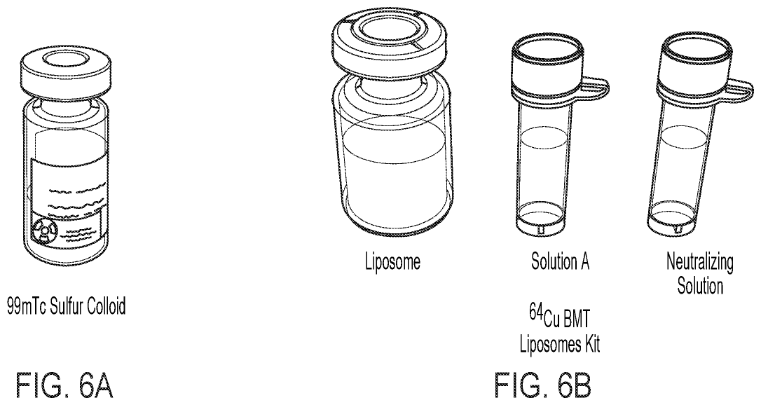

A kit formulation of [⁶⁴Cu]-BMT-LIPO was prepared by the following aseptic procedure (see FIGS. 6A and 6B for identification of kit elements):

(1) Set heating block or heating bath to 50 CC;

(2) Aseptically transfer 125 μL of 500 mM Ammonium Acetate buffer (pH 5.3) (“Solution A”) to BMT-LIPO “Liposome” vial;

(3) Mix the solutions by gentle swirling or vortex;

(4) Add aseptically a desired activity (e.g, in the range of 10mCi-5Ci) of [⁶⁴Cu]—CuCl₂ into BMT-LIPO “Liposome” vial;

(5) Mix the solutions by gentle swirling or vortexing;

(6) Place BMT-LIPO “Liposome” vial in 50° C. heating block or heating bath for 30 minutes;

(7) Aseptically add 32 μL of 500 mM Potassium Carbonate (“Neutralizing Solution”) to BMT-LIPO “Liposome” vial;

(8) Mix the solutions by gentle swirling or vortex; and

(9) Perform Instant Thin Layer Chromatography (iTLC) with 5 mM diethylenetriaminepentaacetic acid (DTPA) as a developing solution if required. Free ⁶⁴Cu peak will be front line and [⁶⁴Cu]-BMT-LIP peak will show up at the origin (point of application).

It is noted that the liposomal formulations described herein do not require a radiolabel in order to function as a delivery vehicle for drugs (e.g., radioprotectants or radiation mitigator such as GT3). However, the liposomal compositions described herein can be radiolabeled to non-invasively track the distribution of the liposome in the body of a subject.

In this example, 500 mM Potassium Carbonate is used as the neutralizing solution. However, other neutralizing solutions can be used in accordance with embodiments of the present disclosure. For example, any solution that raises the pH of the radiolabeled liposomes to approximately pH 7.0-7.4 (e.g., for injection into the subject) is suitable for use with embodiments of the present disclosure.

Furthermore, in this example, 500 mM Ammonium Acetate buffer (pH 5.3) is used as solution A. However, other solutions may be used as solution A in accordance with embodiments of the present disclosure. For example, any solution that reduces the pH of the reaction mixture that enhances the labeling efficiency is suitable for use with embodiments of the present disclosure.

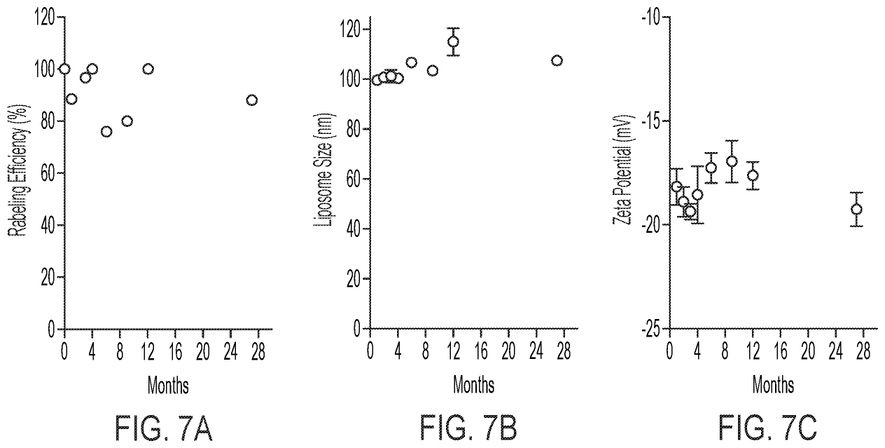

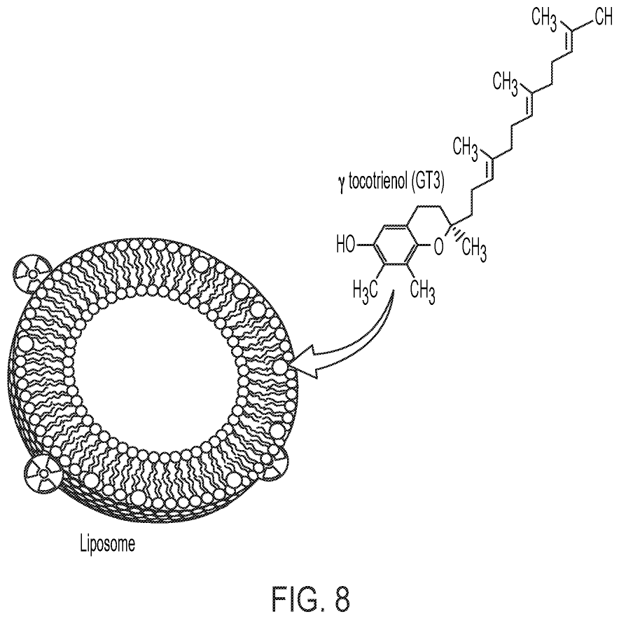

FIGS. 7A-7C show plots representative of the stability of a ⁶⁴Cu BMT Liposomes Kit. BMT-Lipo was stored at 4° C. for 28 months to test stability. Labeling efficiency (FIG. 7A), liposome size (diameter, nm) (FIG. 7B), and zeta potential at pH 7.4 (FIG. 7C) was tested. No significant changes in these measurements were seen over the 28 month period. FIG. 8 shows a schematic of a GT3-loaded liposome in accordance with an embodiment of the present disclosure. GT3-Nano is a non-radioactively labeled formulation that uses the BMT-Lipo platform to encapsulate the radioprotectant GT3. In certain embodiments, the GT3-Nano formulation can be radiolabeled to non-invasively track the distribution of the liposome in the body of a subject. In certain embodiments, the loaded liposome can provide radiation protection in a variety of applications such as external beam and targeted radiation therapy, space travel, and radiation emergencies.

The GT3 liposomal formulation used at AFFRI was equally active as GT3-Nano kit formulations made and used at MSKCC when used to protect mice from death by whole body radiation.

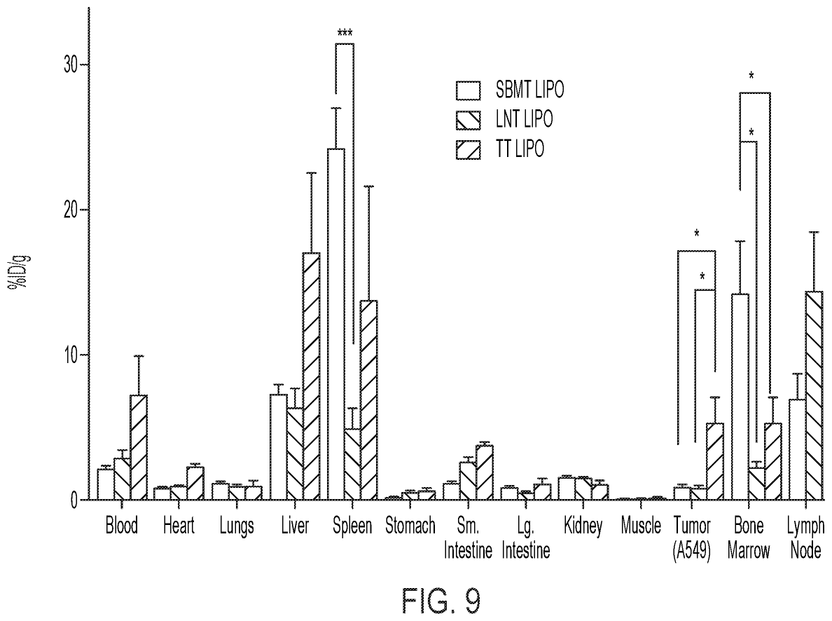

FIG. 9 shows a graph depicting biodistribution of [⁶⁴Cu] labeled selective targeting liposomes at 24 h post injection. SBMT: Spleen, Bone Marrow Targeting; LNT: Lymph Node Targeting; TT: Tumor Targeting. Athymic nude mice bearing A549 tumor were injected with about 140 uCi (5.18 MBq) of ⁶⁴Cu labeled liposomes. 100 ug (2 umoles) of lipid was injected into each mouse through a tail vein injection, which corresponds to approximately 6×10¹² liposome particles per mouse. Mice were euthanized at 24 h post injection and organs were harvested in pre-weighed tubes to measure organ weight and g-counting. N=5 per group; Data is presented as mean and SEM; *p<0.05, ***p<0.001.

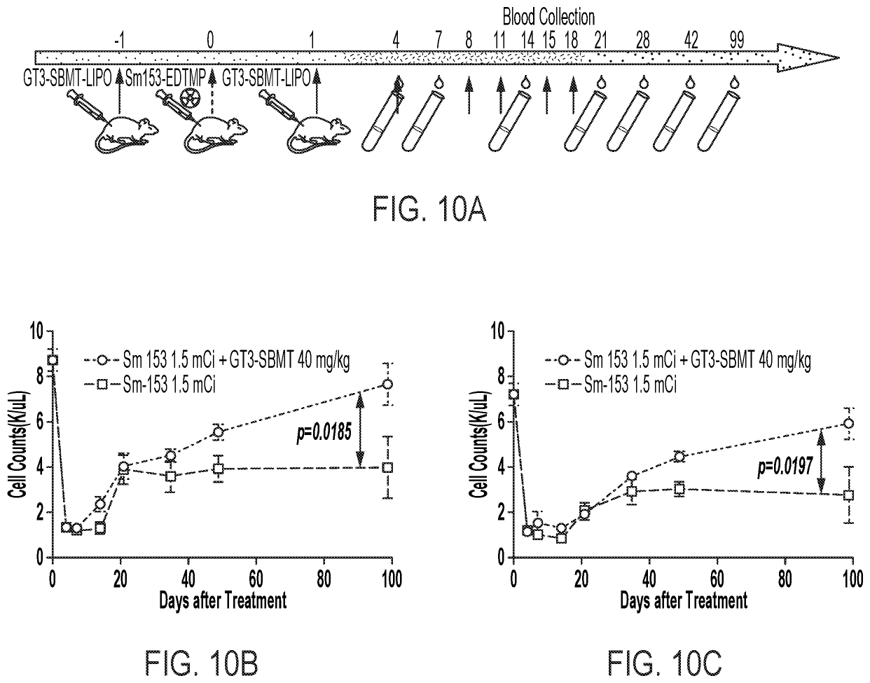

FIGS. 10A-10C show a schematic (FIG. 10A) and plots (FIGS. 10B-10C) that depict white blood recovery of [Sm-153]-EDTMP treatment with SBMT-GT3-LIPO.

FIG. 10A shows a schematic of a [Sm-153]-EDTMP and SBMT-GT3-LIPO injection schedule and blood collection scheme, according to an illustrative embodiment of the present disclosure. FIG. 10B shows a plot depicting white blood cell (WBC) counts (K/uL) at days 4, 7, 14, 21, 28, 42 and 99 after treatment.

FIG. 10C shows a plot depicting lymphocyte counts (K/uL) at days 4, 7, 14, 21, 28, 42 and 99 after treatment.

1.5 mCi of Sm-153 was administered to two groups (5 mice per group) of mice via i.v. 40 mg/kg SBMT-GT3-LIPO was administered 24 h prior to [Sm-153]-EDTMP injection and additional 40 mg/kg SBMT-GT3-LIPO were administered at day 1, 4, 8, 11, 15, and 18. Blood was collected on day 4, 7, 14, 21, 28, 42 and 99. The data was presented as mean±SEM.

GT3 releases from liposomes have critical impact on biodistribution of liposomal drug and its efficacy as a drug. As a highly hydrophobic drug, GT3 was incorporated into the lipid bilayer of liposomes instead of encapsulation inside of the lipid bilayer.

GT3 was originally labeled with ¹³¹I using 1,3,4,6-tetrachloro-3α,6α-diphenyl-glycoluril (Iodogen) because of its ease of labeling GT3 and availability. However, it was found that iodinated GT3 release from BMT-LIPO is faster than GT3 identified by [³H]-GT3, possibly due to its structural changes of iodinated GT3 (data not shown). Data showed that 80% of GT3 was retained in the GT3-BMT-LIPO over 144 h at 6-20 mol % GT3 contents in GT3-BMT-LIPO.

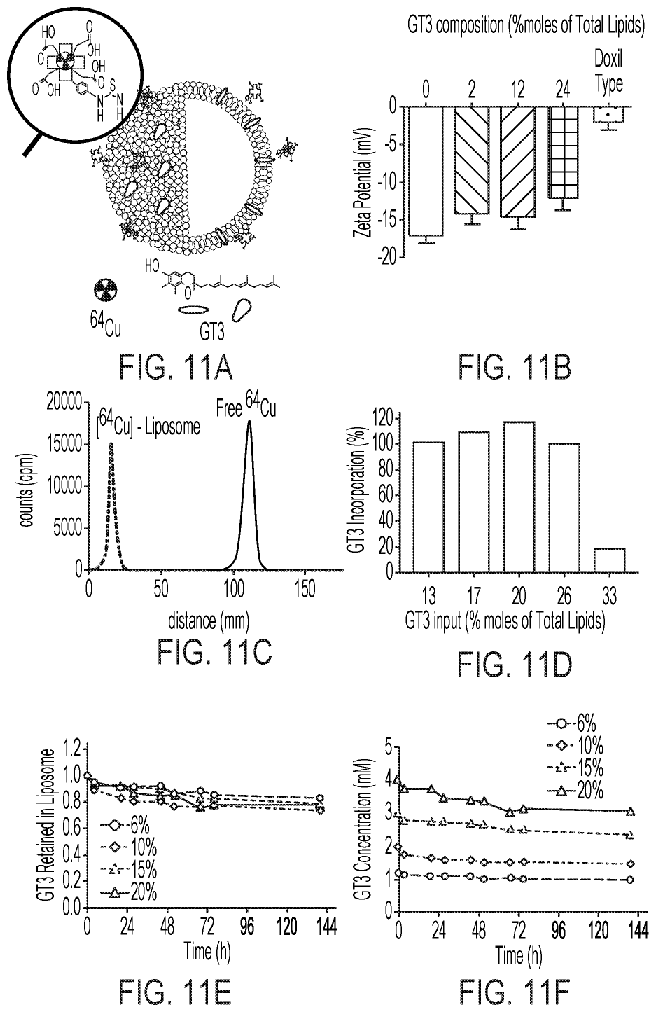

Mechanical filtration, membrane fusion, and the interaction with serum proteins and their cellular response influence the distribution of liposomes in organs and tissues such as bone marrow, liver, spleen, and others. Unlike encapsulated drugs in liposome such as liposomal 11borubicin (Doxil®) and liposomal cytarabine (Depocyt®), GT3 incorporates in the lipid bilayer creating changes of physicochemical properties of liposome lipid bilayer similar to liposomal paclitaxel (LEP-ETU) and liposomal verteporfin (Visudyne®). For example, the size distribution and zeta potential of SBMT-GT3-LIPO can vary. FIGS. 11A-11F confirmed the variation between SBMT-GT3-LIPO (FIGS. 11A-11F).

While SBMT-GT3-Lipo showed high accumulation in the bone marrow, there is moderate accumulation of BMT liposomes in the tumor due to EPR effect. As shown in Table 2, the ratio between bone marrow accumulation and tumor accumulation of SBMT-GT3-Lipo measured by ⁶⁴Cu is 2.7 and 0.7% ID/g at 24 and 48 h respectively. Though GT3 accumulation in the tumor is moderate, there is possibility that GT3 in the tumor might protect tumor during radiation therapy. On the contrary, there are several reports that tocotrienols suppress the proliferation of a wide variety of tumor cells in culture, such as MDA-MB-435, MCF-7, PC-3, LnCaP and DLD-1. Animal studies also have been shown that tocotrienol suppresses the growth of tumors such as 7,12-dimethylbenz(a)anthracene induced mammary tumor, HepG2, and PC-3. To address the question that GT3 protects tumors from irradiation, focal radiation therapy was performed to evaluate the effect of GT3 in the tumor when 10 mg/kg GT3 was administered prior to 11 Gy focal irradiation. Surprisingly, the results showed that GT3 does not protect nor sensitize MDA-MB-468 tumor from 11 Gy focal radiation at 10 mg/kg GT3.

TABLE 2

| | ³H | | | | ⁶⁴Cu | | | |
|---|---|---|---|---|---|---|---|---|
| | 24 h | | 48 h | | 24 h | | 48 h | |
| Blood | 14.97 | (8.85) | 7.54 | (4.81) | 4.98 | (0.25) | 0.50 | (0.07) |
| Tumor(PC9) | N/A | | N/A | | 2.70 | (0.36) | 0.74 | (0.28) |
| Heart | 3.27 | (0.47) | 2.98 | (1.02) | 1.42 | (0.12) | 0.62 | (0.04) |
| Lungs | 3.07 | (1.48) | 3.97 | (1.45) | 1.72 | (0.43) | 0.74 | (0.13) |
| Liver | 6.53 | (1.35) | 6.28 | (0.63) | 14.40 | (1.10) | 9.38 | (0.92) |
| Spleen | 7.95 | (1.61) | 8.26 | (1.66) | 22.86 | (8.14) | 19.20 | (5.00) |
| Stomach | 2.09 | (0.2) | 2.11 | (0.45) | 0.20 | (0.10) | 0.58 | (0.41) |
| Sm. Intestine | 1.6 | (0.3) | 1.66 | (0.28) | 1.94 | (0.54) | 1.74 | (0.39) |
| Lg. Intestine | 1.87 | (0.35) | 2.61 | (0.66) | 0.60 | (0.35) | 0.85 | (0.41) |
| Kidney | 2.59 | (0.46) | 2.41 | (0.33) | 2.06 | (0.51) | 0.72 | (0.25) |
| Muscle | 1.53 | (0.46) | 1.93 | (0.39) | 0.14 | (0.04) | 0.00 | (0.00) |
| Marrow | 4.29 | (3.22) | 7.44 | (2.52) | 12.48 | (2.68) | 6.98 | (2.34) |

SBMT-GT3-Lipo Mitigates Radiation Effects by Supporting Faster and More Complete Recovery of WBC and Lymphocyte Count after Sub-Lethal Total Body Irradiation and [153Sm]-EDTMP Injection.

The present disclosure confirms that GT3 in bone marrow targeting liposome is effective in stimulating bone marrow to increase white blood cell population at 10 mg/kg via i.v., which is considerably lower concentration than 200 mg/kg via s.c.

### Example

The present example describes synthesis, physiochemical characterization, manufacturing, and stability of exemplary compositions (e.g., pre-packaged drug formulations) in accordance with the embodiments of the present disclosure. For instance, the present example describes and tests a composition comprising liposomal carrier of gamma-tocotrienol (GT3), a radioprotectant drug that is a form of Vitamin E (“Vit E”), with enhanced selectivity for spleen bone marrow targeting (SBMT). The liposomal carrier described by the present example is referred to as SBMT-GT3-LIPO. Other radioprotectant drugs can be used.

In certain embodiments, SBMT-GT3-LIPO comprises a PET isotope copper-64 (⁶⁴Cu) as a radiotracer; however, a radiotracer is not required. The present example describes and tests SBMT-GT3-LIPO having a high concentration of gamma-tocotrienol (GT3) (although other concentrations of GT3 can be used in accordance with embodiments of the present disclosure). In certain embodiments, the SBMT-GT3-LIPO can be formulated to comprise a phosphatidyl choline base, be negatively charged, have about a 90 nm diameter, 1% Polyethylene glycol, comprise GT3 concentrations with a range from about 5-20% moles GT3 of total lipids (SBMT-GT3-LIPO). In a certain embodiment, this would mean GT3 comprises 20% of the total number of moles of lipids comprising the liposome, while Succiniyl-PE, cholesterol, mPEG-DSPE, and DSPC comprise the remaining 80% of the total lipids by moles.

### Results

### Quality Characteristics of SBMT-GT3-Lipo Formulations and GT3 Release Kinetics of 3H Labeled SBMT-GT3-Lipo

In certain embodiments, GT3 can be incorporated into bone marrow targeting liposomes at up to 25 mol % of the total liposome. ⁶⁴Cu labeling and stability of the colloidal suspension of GT3-loaded SBMT liposome (SBMT-GT3-Lipo) was measured and found to be substantially similar to bone marrow targeting liposome (FIGS. 11A-11F).

Maximum loading capacity of GT3 in SBMT liposome was determined by adding different amount of GT3 into the fixed amount of SBMT liposome components including: DSPC, cholesterol, Succinyl PE, and mPEG-DSPE (60:40:10:1, molar ratio). Maximum loading capacity, which is defined as where all or nearly all of the GT3 is incorporated into the SBMT liposome, is shown in FIG. 11D. The maximum loading capacity of GT3 into SBMT liposomes was determined to be 26 mol % of GT3 in SBMT liposomes (FIG. 11D).

Size distribution and negative surface charge play the critical role for high accumulation of SBMT liposome in the bone marrow. Therefore, the surface properties, which can be determined through measurements of the zeta potential and size distribution, were determined as GT3 was incorporated into the liposome (FIG. 118). As can be seen from FIG. 11B, GT3 incorporation into the lipid bilayer of SBMT liposomes modifies the zeta potential. Extrusion as a form of size control over the liposome is as effective for SBMT-GT3-Lipo as it is for SBMT liposomes without GT3. The zeta potential as measured at a pH of 7.4 decreased in magnitude by a few millivolts as GT3 was incorporated into SBMT liposomes. As shown in FIG. 11B, zeta potential of 2 mol % SBMT-GT3-Lipo is −14.4 mV. This liposome composition is 2.4 mV less than than zeta potential of SBMT liposome without GT3 at pH 7.4.

Originally, SBMT liposomes were designed to be labeled by ⁶⁴Cu to DOTA-Bn-DSPE in the SBMT liposomes. ⁶⁴Cu was chosen specifically for use with PET imaging. In addition, the radiolabeling efficiency of SBMT liposomes with ⁶⁴Cu was close to 100%. As is shown in FIG. 11C, GT3 incorporation into SBMT liposome does not interfere with labeling of ⁶⁴Cu to DOTA-Bn-DSPE on the SBMT-GT3-Lipo. iTLC showed 100% labeling efficiency of ⁶⁴Cu on the previously mentioned nanoparticles. For example, ⁶⁴Cu binds tightly to DOTA-Bn-DSPE in lipid bilayer of liposome and it does not release from the liposome up to 24 h (data not shown).

There was no GT3-BMT-LIPO precipitation or aggregation observed by visual inspection up to 20 mol % GT3 for several weeks. It was also confirmed that there was no size distribution change by dynamic light scattering.

In vitro GT3 release from SBMT-GT3-Lipo was studied using [³H]-GT3. Release of ³H activity of [³H]-GT3-BMT-LIPO was studied while dialyzed against PBS at different time points up to 140 h at room temperature. In vitro GT3 release was conducted to evaluate physical stability of GT3 in SBMT-GT3-Lipo and tritium labeled GT3 was used to evaluate bioavailability and release kinetics. As shown FIGS. 11E and 11F, the [³H]-GT3 release rate was measured by dialysis against PBS and 73-83% of [³H]-GT3 was retained in the liposome over 140 h of dialysis at room temperature.

### Biodistribution of 64Cu GT3-SBMT-Lipo and 3H Labeled GT3 Showed Similar Accumulation Pattern in the Spleen and Bone Marrow

⁶⁴Cu labeled liposomes were used for tracking intact liposomes. The biodistribution of the ⁶⁴Cu labeled liposomes (without GT3) was compared with the biodistribution of the ⁶⁴Cu- and ³H-labeled GT3 containing liposomes, as an indicator of stability of GT3 drug formulations during in vivo biodistribution and uptake to organs of the MPS.

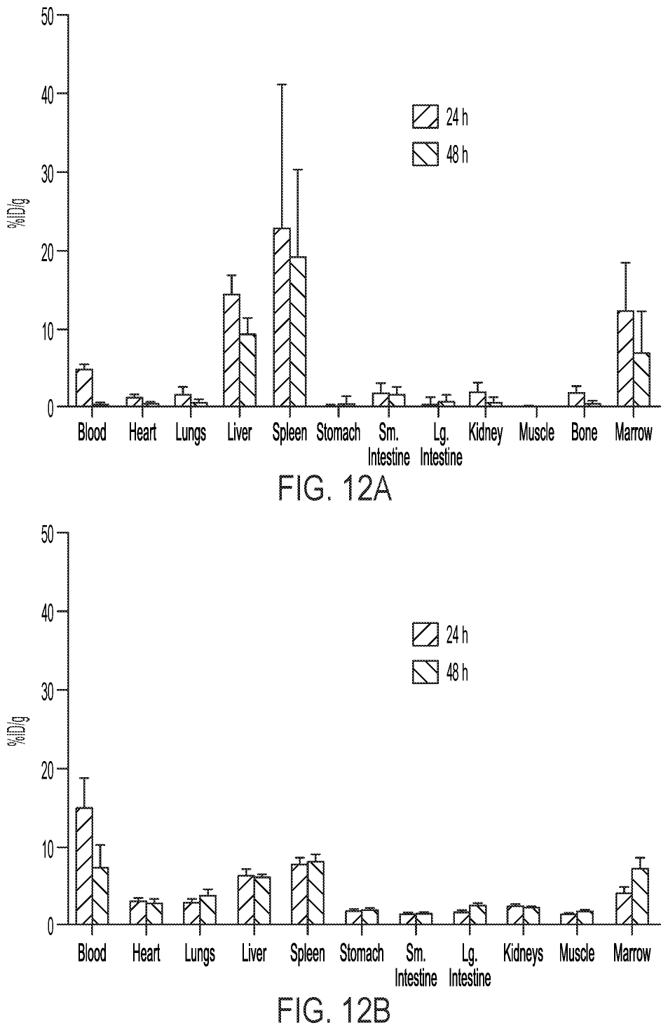

As shown in FIGS. 12A-12B and Table 2, [³H]-GT3 distribution shows the active drug distribution in organs, with approximately 50% loss during the tissue targeting process. Biodistribution data in C57/BL6 mice (n=5 per group) shows that both ³H labeled and ⁶⁴Cu labeled liposomes have a similar biodistribution pattern. The ex vivo biodistribution data of [⁶⁴Cu]-labeled SBMT-GT3-Lipo and [3H]-SBMT-GT3-Lipo at 24 h and 48 h, respectively, is presented. FIGS. 12A-12B show that bone marrow accumulation of SBMT-GT3-Lipo at 24 h is 12.48±2.68% ID/g and at 48 h is 6.98±2.34% ID/g. FIGS. 12A-12B show that spleen and liver are other major organs that SBMT-GT3-Lipo preferentially accumulate. For example, SBMT-GT3-Lipo uptake to spleen was 22.86±8.14% ID/g at 24 h and 19.20±5.00% ID/g at 48 h post administration, respectively. Liver uptake of SBMT-GT3-Lipo was 14.40±1.1% ID/g at 24 h and 9.38±0.92% ID/g at 48 h, respectively. SBMT-GT3-Lipo level in blood was 4.98±0.25% ID/g at 24 h, and it dropped to 0.50±0.07% ID/g at 48 h.

FIGS. 12A-12B show that there are differences in ex vivo biodistribution of SBMT-GT3-Lipo measured by between ⁶⁴Cu and ³H. Accumulations of GT3 in major organs measured by [³H]-GT3 are lower in liver and spleen than accumulations of GT3-BMT-LIFO and higher in other organs measured by ⁶⁴Cu as shown in FIGS. 12A-12B. Marrow uptake of [³H]-GT3 was 4.28±1.57% ID/g and 7.44±2.81% ID/g at 24 and 48 h, respectively, while the uptake of [⁶⁴Cu]-BMT liposome was 12.48±2.68% ID/g and 6.98±2.34% ID/g, respectively. Spleen uptake of [³H]-GT3 was 7.95±1.63% ID/g and 8.26±1.86% ID/g at 24 and 48 h, respectively, while the uptake of [⁶⁴Cu]-BMT liposome was 22.86±8.14% ID/g and 19.20±5.00% ID/g, respectively.

Table 2 (as seen in the section entitled “Kit Formulation”) shows the biodistribution of GT3-loaded bone marrow targeting liposomes labeled with [³H]-GT3 and [⁶⁴Cu]-DOTA-Bn-DSPE. 5.5 MBq of ⁶⁴Cu labeled SBMT-GT3-Lipo and 370 kBq of ³H labeled SBMT-GT3-Lipo were injected to mice via the tail vein (n=5 per group). Mice were sacrificed at the indicated time and major organs were collected and weighed. ⁶⁴Cu counting was done as soon as the organs were collected and ³H counting was measured 5 days after mice were sacrificed. The % ID/g was calculated by measuring weight and time corrected measurement of radioactivity. n=5 per group.

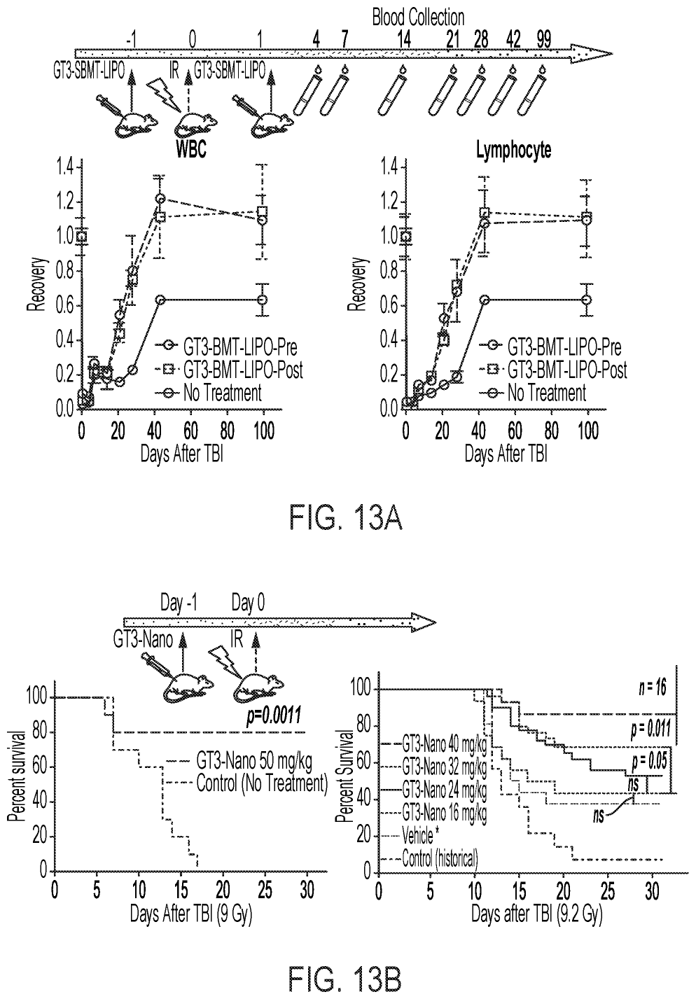

As shown in FIGS. 13A, 13B, and 10A-C, 4 Gy whole body radiation induced severe depletion of peripheral blood cells. White blood cells (WBC), lymphocytes, and neutrophils decreased to less than 10% of baseline counts with a nadir at day 1-4, post irradiation. Recovery of WBC and lymphocytes in both GT3 pre-treated and post-treated groups was faster and more complete than the untreated group. Recovery of GT3 treated groups reached 100% at day 43. For the untreated group, WBC and lymphocyte counts recovered about 60% at day 43 and WBC and lymphocyte counts never recovered to normal level until day 99. Neutrophil counts decreased to nadir on day 4 for GT3 treated and untreated groups. The platelet counts recovered on day 7 and remained relatively the same counts until day 99. Platelet counts increased on day 1 and decreased to nadir on day 14. Platelet counts were recovered to normal level at day 43 and remained the same level until day 99 in all groups. There were no significant differences in platelet counts between GT3 treated and untreated groups at 4 Gy TBI.

To test SBMT-GT3-LIPO ability to protect bone marrow from internal and continuous irradiation, mice were treated with [¹⁵³Sm]-EDTMP, a radionuclide which is used to treat pain by bone metastasis of cancers. 40 mg/kg of SBMT-GT3-LIPO was administered 24 h prior to 1.5 mCi [¹⁵³Sm]-EDTMP injection followed by 6 injections of 40 mg/kg SBMT-GT3-LIPO over 3 weeks (day 1 and 4 of each week). As shown in FIGS. 10A-C, GT3 pre-treatment did not prevent the depletion of WBCs and lymphocytes. Accordingly in FIGS. 10B and 10C, the WBC and lymphocyte counts dropped to 15% of baseline at day 4. Recovery of WBCs is delayed until day 7 for both SBMT-GT3-LIPO treated and control group. The nadir continued to day 14 for control group while the WBC counts of SBMT-GT3-LIPO started to recover at day 14. It was found that recovery of WBC in SBMT-GT3-LIPO treated group was faster and reached 88% over 99 days while recovery of WBC counts in control group reached 44% at day 21 and the recovery did not improve until day 99.

SBMT-GT3-Lipo Increased Survival of Mice from Lethal Total Body Irradiation, when Administered 24 Hours Prior to Irradiation, in a Dose Dependent Manner.

To evaluate the efficacy of SBMT-GT3-Lipo at lethal dose of total body irradiation, C57/B6 (FIG. 13B) were first tested after irradiation of 9 Gy of Cesium-131 irradiation delivered over 12 minutes. 50 mg/kg SBMT-GT3-LIPO treatment to C57/B6 mice showed 80% survival at day 30 while untreated group showed no mice survival at Day 17 at 9.0 G.

To test the dose dependency of survival with SBMT-GT3-LIPO treatment, CD2F1 mice were treated with 16, 24, 32, 40 mg/kg SBMT-GT3-LIPO and SBMT-LIPO as a control. The mice survival at 16 and 24 mg/kg group did not show any statistical significance compared to control group. However, the mice survival of 32 mg/kg treatment group at day 30 was 67% (p<0.05) to control group and the mice survival of 40 mg/kg at day 30 group was 87% (p<0.05). Thus, it was confirmed that SBMT-GT3-LIPO can rescue mice of two different strains from otherwise lethal radiation in a dose dependent manner.

CD105 Colocalization with Fluorescence Labeled SBMT-LIPO

4-amino-7-nitrobenz-2-oxa-1,3-diazole (NBD)-SBMT-LIPO was formulated to investigate the localization of SBMT-LIPO in the spleen. NBD labeling to SBMT-LIPO was achieved by adding 0.5 μg NBD-PE in 20 μmoles of total lipid mixture for SBMT-LIPO. Size distribution and zeta-potential NBD-SBMT-LIPO are the same as SBMT-LIPO by dynamic light scattering method (data not shown).

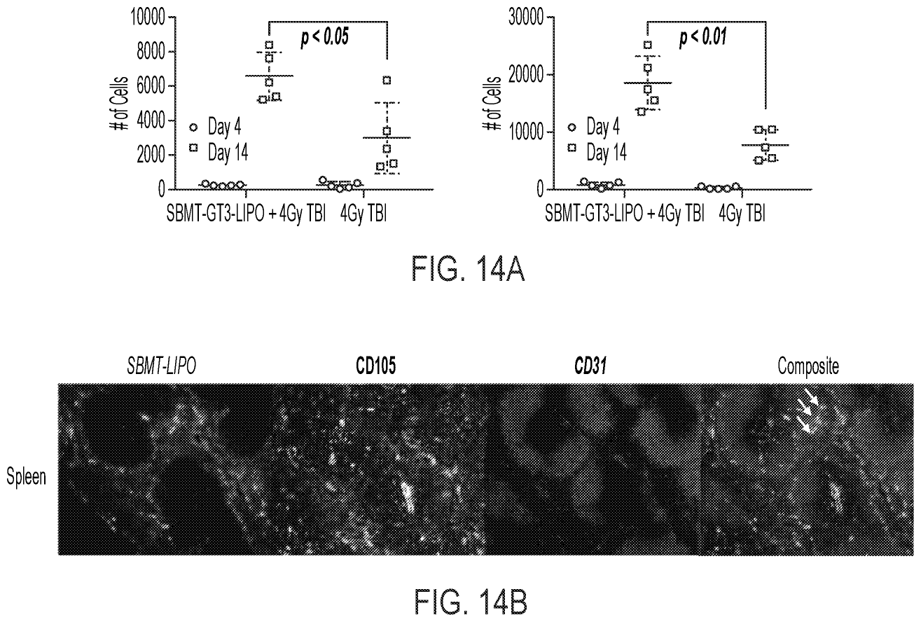

As shown in FIG. 14B, NBD-SBMT-LIPO is primarily localized in endothelial cells lining the sinusoids, within the red pulp region in the spleen. In the red pulp region, the sinusoids are engorged with blood and macrophages. Anti-CD105 and anti-CD31 antibodies were used to identify the degree to which endothelial cells phagocytosed liposomal particles and NBD-SBMT-LIPO. CD105 and CD31 are markers for endothelial cells known to produce the lymphokine IL33, which actives HSC in the nearby stem cell niche, driving cells toward hematopoiesis, angiogenesis and osteogenesis. Anti-CD105 antibodies show up as bright, high intensity spots within vascular and sinusoidal endothelial cells, activated macrophages and activated monocytes. In addition, NBD-SBMT-LIPO colocalization with CD105 is seen within these cells. CD105+ cells are mainly localized in red pulp region. However, some CD105+ cells are found within the white pulp region where lymphocytes are abundant. CD31+ has been found on endothelial cells, platelets, macrophages, Kupffer cells, and lymphocytes. As shown in the FIG. 14B, CD31+ cells were primarily located in the marginal zone of the spleen which is interface between the non-lymphoid red pulp and B cell abundant white pulp of the spleen. However, NBD-SBMT-LIPO did not colocalize with CD31+ cells with few exceptions. A composite image of NBD-SBMT-LIPO, CD105+, and CD31+ shows that NBD-SBMT-LIPO is colocalized with CD105+ cells in the red pulp region. CD31+ is only colocalized with NBD-SBMT-LIPO in CD31+/CD105+ cells.

Flow Cytometry Shows Faster and More Complete Recovery of the Precursor Hematopoietic Cell Populations (MMP2 and CMP) in Bone Marrow, after Injection of SBMT-GT-3 Lipo Nanoparticles.

GT3 is an inhibitor of hydroxyl-methyl-glutaryl-coenzyme A reductase (HMGCR) and induces production of G-CSF and expression of DNA repair gene RAD50 and other unknown mechanisms. While CD105+ cells of the endothelium uptake GT3-SBMT-LIPO, it is unclear which cell population is protected by GT3-SBMT-LIPO in bone marrow.

Hematopoietic stem cells (HSCs) and multipotent progenitors (MPPs) are responsible for the replenishing other blood cells through the hematopoietic process, although HSCs mostly exist in a state of quiescence, or reversible growth arrest. The altered metabolism of quiescent HCSs helps the cells survive for extended periods of time. When provoked by cell death or damage, HSCs exit quiescence and begin actively dividing again. There are a number of markers that permit the prospective identification and isolation of HSCs and MPPs. Changes to HSCs and MPPs in bone marrow were analyzed to identify which subpopulations of HSCs/MPPs are protected by GT3-SBMT-LIPO treatment. As shown in the FIG. 14A, myeloid-biased MPP subsets 2 (MPP2) and common myeloid progenitors (CMP) showed statistically significant recovery in comparison to other HSC subpopulations at days 4 and 14.

### Materials and Methods

### Materials

All chemicals were used as received without further purification. 1,2-distearoyl-sn-glycero-3-phosphocholine (DSPC), 1,2-dioctadecanoyl-sn-glycero-3-phosphoethanolamine(DSPE), 1,2-dipalmitoyl-sn-glycero-3-phosphoethanolamine-N-(succinyl) (Succinyl-DPPE), and 1,2-distearoyl-sn-glycero-3-phosphoethanolamine-N-[methoxy(polyethylene glycol)-2000] (mPEG2000-DSPE); these, along with cholesterol, were purchased from Avanti Polar Lipids (Alabaster, Ala.). The p-SCN-Bn-DOTA was purchased from Macrocyclics (Dallas, Tex.), and ⁶⁴Cu was purchased from Washington University in St. Louis (St. Louis, Mo.). Gamma-tocotrienol (GT3) was purchased from Chromadex (Irvine, Calif.) and tritium labeled GT3 was synthesized at ViTrax (Placentia, Calif.). Mini extruder was purchased from Avanti Polar Lipids (Alabaster, Ala.) and 10 mL LIPEX™ thermobarrel extruder was purchased from Transferra NanoScience Inc. (Burnaby, British Columbia, Canada). PD-10 column was purchased from GE Healthcare Life Sciences (Pittsburgh, Pa.), and athymic nude mice were purchased from Envigo Laboratories (Indianapolis, Ind.).

### Synthesis of DOTA-Bn-DSPE

12 μmoles of p-SCN-Bn-DOTA was dissolved in 1 mL of chloroform:methanol:water (65:35:8) mixture and 22 μmoles of DSPE was dissolved in 1 mL of the chloroform:methanol:water mixture. After mixing two solutions, 48 μmoles of triethylarnine was added. The mixture was stirred at 40° C. for 2 h followed by stirring at room temperature for 16 h. The reaction progress was monitored using silica gel-coated TLC plates and product formation was confirmed by mass spectroscopy.

### Preparation of Liposomes

Liposomes were composed of DSPC and cholesterol in a molar ratio of 6:4. Depending on the formulation, the initial lipid mixture was supplemented with 1% mol of mPEG2000-DSPE, 10% succinyl-DPPE and 10% gamma-tocotrienol. Additional 0.1% mol DOTA-Bn-DSPE was added to all lipid composition for facilitating subsequent ⁶⁴Cu labeling. All lipids were dissolved in chloroform and the solvent was evaporated under flowing nitrogen gas at 37° C. Residual solvent was removed under vacuum (0.2 Torr) for at least 2 h. Lipid film was hydrated in PBS at 65° C. for 1 h and the crude lipid dispersion was extruded 11 times at 65° C. through 0.1 μm or 0.03 μm pore size Whatman® Polycarbonate Membrane Filter using mini extruder system or LIPEX™ thermobarrel extruder. After extrusion, the liposomes were purified using a PD-10 column (GE Life Sciences, Marlborough, Mass.) to remove unincorporated liposomal lipids and salts.

### Characterization of Liposomes

Liposome size distribution and zeta potential at 25° C. in PBS pH 7.4 were determined by dynamic light scattering using Zetasizer Nano-ZS from Malvern Instruments (Malvern, Worcestershire, UK). Liposome stability under serum was determined after incubation in 50% FCS in PBS for 24 h. Long-term liposome stability was tested by maintaining the liposome at 4° C. for two years and analyzing size distribution.

Radioactive Labeling of Liposome with 64Cu and 131/124I

[⁶⁴Cu]—CuCl₂ (750 μCi in 0.1 N HCl) was added to 750 μL of 20 μM total lipid concentration liposomes and adjusted to pH 5.5 with 0.2 M sodium acetate buffer (pH 5.5). The reaction mixture was stirred at 50° C. for 1 h with constant shaking using an Eppendorf ThermoMixer®.

ITLC was performed on an ITLC-SG paper using 5 mM DTPA solution (pH 5.5) as an eluent to monitor the progress of reaction. To label GT with ¹³¹I, 100 μCi [^{131/124}I]—NaI was added to 750 μL of 20 μM total lipid concentration liposomes in 1,3,4,6-tetrachloro-3α,6α-diphenyl-glycoluril coated tube for 10 mins at room temperature and PD-10 column was used to separate liposomes from unreacted [^{131/124}I]—NaI.

In Vitro GT3 Release from the Liposomes

2 mL of 4 μCi³H labeled GT3 containing BMT liposomes was incubated inside dialysis cassette (Pierce Slide-A-Lyzer Dialysis cassett with a MWCO 50 k) in PBS for 96 h and 1004 of the liposomes were taken, mixed with 5 mL of Ecoscint A at 0, 4, 20, 28, 44, 52, 68 and 140 h and measured the activity using scintillation counter. The accumulative release curve was built as the percentage of release (% R) at each assayed time applying % R=(R/T)×100 where, R represents the activity measured in the collected sample and T represents the total amount of activity measured at the beginning of the experiment.

Biodistribution of ⁶⁴Cu Liposome and ³H Labeled GT3 Containing Liposome

For biodistribution studies, approximately 5.5 MBg of [⁶⁴Cu]-DOTA-Bz-DSPE-labeled liposome and 370 kBq of ³H-GT3 incorporated liposome was intravenously administered to C57/BL6 athymic nude mice (n=5 per group) via tail vein injection. Mice were sacrificed at 24 or 48 h after injection and major organs were collected and placed in pre-weighed culture tubes or Eppendorf tubes. To collect bone marrow, the femur was dipped into liquid nitrogen and one of the epiphyses was carefully removed. A 30 mL syringe with gauge 30½ needles was inserted into one end of the femur; air was blown and bone marrow was collected in the same tubes for radioactivity counting in a gamma counter (PerkinElmer, Inc., Waltham, Mass.). For ³H activity measurement, major organs were collected in pre-weighed scintillation vials and 500 uL of Soluene®-350 were added. After overnight incubation at 37° C., 4.5 mL of Ecoscint A were added, mixed well, and counted in scintillation counter. Data were presented as percent injected dose per gram (% ID/g) of tissue.

### In Vivo Efficacy of GT3 Liposomes Against Total Body Irradiation

6- to 8-week-old C57/BL6 mice (Jackson Lab., Bar Harbor, Me.) were used for bone marrow protection from total body irradiation studies. 10 mg/kg GT3 liposomes (6% mol) were administered via i.v. 24 h pre- and post-total body irradiation (4 Gy, Cs-135, dose rate=82 cGy/min). No supportive care was given to mice after irradiation. Approximately 100 μL of blood was collected by retro-orbital blood collection for complete blood cell counts at various time points for 100 days.

### Immunohistochemistry

7 week old C57/BL6 mice were injected with 100 μL of NBD-BMT-LIPO. The spleens were collected into optimal cutting temperature (OCT) compound at 4 h post injection. The spleens were subsequently frozen at a temperature of approximately −78° C. The frozen spleen section block was cut at 10 μm thickness using an Avantik Cryostatic Microtome. The sections were then washed 3 times with PBS and blocked with TBS+1% BSA solution for 2 hours. Sections were subsequently incubated with mouse anti-CD31 IgG (20 μg/mL) and rabbit anti-CD105 IgG (5 μg/mL) overnight at 4° C. After incubation, the unbound primary antibodies were washed off of the sections by using three consecutive PBS washes. The sections were then incubated with goat anti-mouse IgG-Alex-647 and goat anti-mouse IgG-Alexa594 for 2 hours at room temperature. Sections were mounted on slides using fluorescent mounting media containing DAPI and observed under Zeiss Axio Imager 2 Microscope.

### Software and Statistics

GraphPad Prism 7.0 (GraphPad Software Inc., La Jolla, Calif.) was used for plotting graphs, fitting curves, and analyzing statistics. Collected data are presented as mean±SD. ASIPro VM (Siemens Medical Solutions, Knoxville, Tenn.) and Amide²⁷ was used for PET and PET/CT image analysis. ImageJ was used to analyze immunohistochemistry (IHC) images.

### Alternative Embodiments

Implementation 1. A composition comprising: a liposome-based nanocarrier comprising: a lipid; and an organic polymer, wherein the liposome-based nanocarrier has a surface having a negative charge due to the lipid, and an associated drug (e.g., wherein the associated drug is encapsulated inside of the liposome-based nanocarrier, incorporated into layers of the lipid, or covalently and/or non-covalently attached to the lipid on the surface of liposome-based nanocarrier).

Implementation 2. The composition of implementation 1, wherein the associated drug comprises a member selected from the group consisting of: a free radical scavenger (e.g., gamma-tocotrienol (GT3)); a radioprotectant or radiation mitigator (e.g., Amifostine, N-acetylcystein, alpha-tocotrienol, gamma-tocotrienol, delta-tocotrienol, Genistein, rapamycin); a growth factor (e.g., erythropoietin, granulocyte colony-stimulating factor, RNAi therapeutics (e.g., GTI-2040 (ribonucleotide reductase), SPC2996 (Bcl-2), LY2181308 (survivin), e.g., immunosuppressors (e.g., Tacrolimus, mTOR inhibitors, corticosteroids, antibiotics, epinephrine analogs, RNAi against Bim and PUMA)); a bisphosphonate (e.g., alendronate, ibandronate, risedronic acid, zoledonic acid); a selective estrogen receptor modulator (e.g., raloxifene); a parathyroid hormone modulator (e.g., teriparatide); a biological (e.g., denosumab); a chemotherapeutic drug (e.g., any of the chemotherapeutic drugs listed in Table 1); a CXCR2 agonist (e.g., GROb, GROa) (e.g., for mobilizing stem cells); a CXCR4 antagonist (e.g., AMD3100, BIO8020) (e.g., for mobilizing stem cells); a HMG-CoA reductase (also known as 3-hydroxy-3-methyl-glutaryl-coenzyme A reductase or HMGCR) inhibitor (e.g., a statin (e.g. simvastatin, atorvastatin, lovastatin, pitavastatin)) for protecting marrow cells); a superoxide dismutase (SOD) mimetic or other mimetics (e.g., manganese(III) 5,10,15,20-tetrakis(4-benzoic acid)porphyrin (MnTBAP), manganese (III) mesotetrakis (N-ethylpyridinium-2-yl) porphyrin (MnTE-2-PyP(5+)) (AOEL 10113)) (e.g., for removing free radicals); an androstenediol (e.g., for stimulating white blood cells (WBCs) and platelet growth); an androgen receptor (AR) agonist (e.g., dihydrotestosterone (DHT), a nonsteroidal selective androgen receptor (SARM) (e.g., BMS-564,929, VK5211) (e.g., for stimulating production of erythropoietin in the treatment of myeloid metaplasia); and any combination thereof.

Implementation 3. The composition of implementation 1 or 2, wherein the lipid comprises a member selected from the group consisting of 1,2-dioleoyl-sn-glycero-3-phosphoethanolamine-N-(succinyl) (succinyl PE), 1,2-distearoyl-sn-glycero-3-phosphocholine (DSPC), cholesterol, 1,2-distearoyl-sn-glycero-3-phosphoethanolamine (DSPE), and 1,2-dipalmitoyl-sn-glycero-3-phosphoethanolamine-N-(succinyl) (succinyl-DPPE).

Implementation 4. The composition of any one of the preceding implementations, wherein the lipid is labeled with an isotope and chelator.

Implementation 5. The composition of any one of the preceding implementations, wherein the isotope comprises a member selected from the group consisting of 64Cu, 66Ga, 86Y, 111In, 67Ga, 68Ga, 89Zr, 124/131I, and 177Lu.

Implementation 6. The composition of any one of the preceding implementations wherein the chelator comprises a member selected from the group consisting of 1,4,7,10-tetraazacyclododecane-1,4,7,10-tetraacetic acid (DOTA), 1,4,7-triazacyclononane-1,4,7-trisacetic acid (NOTA), and 1-(4-isothiocyanatophenyl)-3-[6,17-dihydroxy-7,10,18,21-tetraoxo-27-(N-acetylhydroxylamino)-6,11,17,22-tetraazaheptaeicosine] thiourea (DFO).

Implementation 7. The composition of any one of the preceding implementations, wherein the chelator comprises a member selected from the group consisting of DOTA-Bn-DSPE, NOTA-Bn-DSPE, and DFO-Bz-DSPE.

Implementation 8. The composition any one of the preceding implementations, wherein the organic polymer comprises polyethylene glycol (PEG).

Implementation 9. The composition of any one of the preceding implementations, wherein the nanocarrier comprises 1,2-distearoyl-sn-glycero-3-phosphoethanolamine-N-methoxy(polyethylene glycol) (mPEG-DSPE).

Implementation 10. The composition of implementation 8 or 9, wherein the concentration of PEG is from about 0.5 wt. % to about 10 wt. %.

Implementation 11. The composition of implementation of any one of the preceding implementations, wherein the liposome-based nanocarrier is at least 3 mole % lipid.

Implementation 12. The composition of any one of the preceding implementations, wherein the liposome-based nanocarrier has an average diameter in a range from 30 nm to 300 nm.

Implementation 13. The composition of any one of the preceding implementations, comprising one or more of (i), (ii), and (iii), as follows: (i) from 3 to 20 wt. % succinyl-DPPE; (ii) from 0.5 to 2 wt. % PEG; and (iii) from 5 to 9 wt. % PEG.

Implementation 14. The composition of any one of the preceding implementations, wherein the negative charge of the surface of the liposome-based nanocarrier has a magnitude from 15 mV to 25 mV.

Implementation 15. A method for imaging a subject the method comprising: administering to the subject a composition of any one of the preceding implementations, wherein the lipid is labeled with an isotope and a chelator.

Implementation 16. The method of implementation 15, further comprising obtaining and displaying a positron emission tomography (PET) and/or Positron emission tomography-computed tomography (PET/CT) image of at least one tissue of the subject comprising the composition.

Implementation 17. The method of implementation 15 or 16, further comprising quantitatively measuring a distribution of the composition in at least one tissue of the subject.

Implementation 18. The method of implementation 17, the method comprising quantitatively measuring the distribution of the composition in an organ of the reticuloendothelial system.

Implementation 19. The method of implementation 18, wherein the organ comprises a member selected from the group consisting of liver, spleen, and bone marrow.

Implementation 20. The method of implementation 18 or 19, comprising determining a concentration and/or total amount of delivered radiolabeled drug in the tissue based on a positron emission tomography (PET) or Positron Emission Tomography-Computed Tomography (PET/CT) image of the tissue.

Implementation 21. The method of any one of implementations 17 to 20, the method comprising quantitatively measuring the distribution of the composition in one or more lymph nodes.

Implementation 22. The method of any one of implementations 15 to 21, wherein the administered composition demonstrates selective targeting of bone marrow of the subject such that concentration of the composition in bone marrow is at least 3 fold greater than the concentration of the composition in any of the tumor tissue at a given time following administration of the composition, wherein the given time is at least 1 hour following administration.

Implementation 23. The method of any one of implementations 15 to 22, further comprising capturing and displaying a sequence of PET images in real time.

Implementation 24. A method of treating a subject, the method comprising administering the composition of any one of implementations 1 to 14 to the subject suffering from or susceptible to a disease and/or condition.

Implementation 25. The method of implementation 24, wherein the disease and/or condition comprises a member selected from the group consisting of bone marrow suppression (BMS), myelodysplastic syndrome (MDS), acute myeloid leukemia (AML), chronic myeloid leukemia (CML), sepsis, graft-versus-host-disease (GVHD), bone metastasis, osteoporosis, and myeloid metaplasia.

Implementation 26. The method of implementation 24 or 25, wherein the disease and/or condition comprises exposure to radiation.

Implementation 27. The method of implementation 25, the method further comprising after administering the composition, administering a chemotherapeutic and/or radiation therapy.

Implementation 28. The method of any one of implementations 24 to 27, wherein the administered composition demonstrates selective targeting of bone marrow of the subject such that the concentration of the liposome-based nanocarrier in bone marrow is at least 3 fold greater than the concentration of the liposome-based nanocarrier in any of the tumor tissue at a given time following administration of composition, wherein the given time is at least 1 hour, following administration.

Implementation 29. The method of any one of implementations 24 to 28, wherein the administered composition protects bone marrow cells against radiation (e.g., wherein the subject has received or will receive radiation exposure, e.g., from a chemotherapy drug, radiation treatment, and/or other radiation exposure).

Implementation 30. The method of any one of implementations 24 to 29, wherein the administered composition mobilizes and recruits stem cells to the bone marrow (e.g., wherein the subject has received or will receive radiation exposure, e.g., from a chemotherapy drug, radiation treatment, and/or other radiation exposure).

Implementation 31. The method of any one of implementations 24 to 30, wherein the administered composition stimulates production of erythropoietin (e.g., wherein the subject has received or will receive radiation exposure, e.g., from a chemotherapy drug, radiation treatment, and/or other radiation exposure).

Implementation 32. A method of monitoring a patient, the method comprising administering the composition of any one of implementations 1 to 14 to a patient suffering from or susceptible to a disease and/or condition; and investigating a quantity of drug (e.g., a drug currently or having been associated with the liposome-based nanocarrier of the composition) delivered to at least one tissue of the patient.

Implementation 33. A method of imaging an organ of the reticuloendothelial system in a subject, the method comprising: detecting radiation from the liposome-based nanocarrier of any one of implementations 1 to 14, the subject having been administered the composition.

Implementation 34. The method of implementation 33, wherein the radiation is detected via an external PET imaging system.

Implementation 35. The method of implementation 33 or 34, wherein the organ comprises a member selected from the group consisting of active bone marrow, liver, and spleen.

Implementation 36. The method of any one of implementations 33 to 35, the method further comprising displaying an image corresponding to the detected radiation, the image visually distinguishing active bone marrow from other tissue and, optionally, quantifying the concentration of drug and/or liposome-based nanocarrier.

Implementation 37. A composition comprising: a liposome-based nanocarrier comprising: a lipid; and an organic polymer, wherein the liposome-based nanocarrier has a surface having a negative charge due to the lipid, and an associated drug (e.g., wherein the associated drug is encapsulated inside of the liposome-based nanocarrier, incorporated into layers of the lipid, or covalently and/or non-covalently attached to the lipid on the surface of liposome-based nanocarrier), for use in a method of treating a disease and/or condition in a subject, wherein the treating comprises delivering the composition to the subject.

Implementation 38. A composition comprising: a liposome-based nanocarrier comprising: a lipid; and an organic polymer, wherein the liposome-based nanocarrier has a surface having a negative charge due to the lipid, and an associated drug (e.g., wherein the associated drug is encapsulated inside of the liposome-based nanocarrier, incorporated into layers of the lipid, or covalently and/or non-covalently attached to the lipid on the surface of liposome-based nanocarrier), for use in a method of monitoring of a disease and/or condition in a subject, wherein the monitoring comprises delivering the composition to the subject.

Implementation 39. A composition comprising: a liposome-based nanocarrier comprising: a lipid; and an organic polymer, wherein the liposome-based nanocarrier has a surface having a negative charge due to the lipid, and an associated drug (e.g., wherein the associated drug is encapsulated inside of the liposome-based nanocarrier, incorporated into layers of the lipid, or covalently and/or non-covalently attached to the lipid on the surface of liposome-based nanocarrier), for use in (a) a method of treating a disease and/or condition in a subject or (b) in a method of monitoring of a disease and/or condition in a subject, wherein the monitoring comprises delivering the composition to the subject.

Implementation 40. A composition comprising: a liposome-based nanocarrier comprising: a lipid; and an organic polymer, wherein the liposome-based nanocarrier has a surface having a negative charge due to the lipid, and an associated drug (e.g., wherein the associated drug is encapsulated inside of the liposome-based nanocarrier, incorporated into layers of the lipid, or covalently and/or non-covalently attached to the lipid on the surface of liposome-based nanocarrier), for use in therapy.

Implementation 41. A composition comprising: a liposome-based nanocarrier comprising: a lipid; and an organic polymer, wherein the liposome-based nanocarrier has a surface having a negative charge due to the lipid, and an associated drug (e.g., wherein the associated drug is encapsulated inside of the liposome-based nanocarrier, incorporated into layers of the lipid, or covalently and/or non-covalently attached to the lipid on the surface of liposome-based nanocarrier), for use in monitoring a disease or condition.

Implementation 42. A kit (e.g., for use in radioprotection or radiation mitigation, e.g., for use in methods of treating, monitoring, and/or imaging a subject and/or patient) comprising: At least one container (e.g., an ampule, a vial, a cartridge, a reservoir, a lyo-ject, or a pre-filled syringe); and a composition of any one of implementations 1 to 14 or 37 to 41.

Implementation 43. The kit of implementation 42, comprising a neutralizing solution (e.g., wherein the neutralizing solution is contained in a first one of the at least one containers) (e.g., wherein the neutralizing solution adjusts the pH to about pH 7.0-7.4) (e.g., wherein the neutralizing solution comprises potassium carbonate, e.g., 500 mM potassium carbonate).

Implementation 44. The kit of implementation 42 or 43, comprising a Solution A (e.g., wherein the Solution A is contained in a second one of the at least one containers) (e.g., wherein the Solution A enhances labeling efficiency, e.g., wherein Solution reduces pH) (e.g., wherein the Solution A comprises ammonium acetate, e.g., 500 mM ammonium acetate).

Implementation 45. The kit of any one of implementations 42 to 44, comprising a radiolabel (e.g., 64Cu, e.g., 64Cu—Cl2, e.g., 89Zr) (e.g., wherein the radiolabel is contained in a third one of the at least one containers).

Implementation 46. A method for preparing a composition (e.g., a composition of any one of implementations 1 to 14 or 37 to 41), the method comprising: contacting (e.g., via gentle swirling and/or mixing) a first solution with a substance comprising a radiolabel (e.g., 64Cu, e.g., 64Cu—Cl2, e.g., 89Zr) to generate a second solution (e.g., wherein the average labeling yield is greater than 70%, greater than 80%, greater than 90%), wherein the first solution comprises a liposome-based nanocarrier comprising: a lipid, and an organic polymer, wherein the liposome-based nanocarrier has a surface having a negative charge due to the lipid; heating (e.g., at 50° C.) the second solution for a period of time (e.g., for about 10 minutes, for about 20 minutes, for about 30 minutes, for about 40 minutes); and contacting (e.g., aseptically) (e.g., via gentle swirling and/or mixing) the second solution with a Neutralizing Solution to generate a third solution (e.g., thereby adjusting pH of the third solution to a value of about 7.0 to about 7.4) (e.g., wherein the third solution comprises the composition of any one of implementations 1 to 14 or 37 to 41) (e.g., wherein the third solution has a pH value of from about 7.0 to about 7.4).

Implementation 47. The method of implementation 46, the method further comprising: contacting (e.g., aseptically) (e.g., via gentle swirling and/or mixing) a Solution A with the liposome-based nanocarrier, thereby generating the first solution [e.g., enhances efficiency particularly where radiolabel comprises 64Cu] wherein the Solution A enhances radiolabeling efficiency, e.g., wherein Solution A reduces pH of the first solution (e.g., wherein the Solution A comprises ammonium acetate, e.g., 500 mM ammonium acetate)].

Implementation 48. The method of implementation 46 or 47, wherein the Neutralizing Solution comprises potassium carbonate, e.g., 500 mM potassium carbonate.

What is claimed is:

1. A method of treating, monitoring, and/or imaging a subject, the method comprising:
receiving a kit (e.g., via mail, e.g., via courier), the kit comprising a first container (e.g., an ampule, a vial, a cartridge, a reservoir, a lyo-ject, or a pre-filled syringe), wherein the first container contains a composition, said composition comprising (i) a lipid; (ii) an organic polymer; and (iii) gamma-tocotrienol (GT3) (e.g., and/or an equivalent or derivative of GT3), wherein the liposome-based nanocarrier has a surface having a negative charge;
storing the kit (e.g., storing the kit at about 4° C.) for a storage duration; and
administering the kit to a subject after the storage duration [e.g., wherein the storage duration is at least 2 weeks (e.g., at least 1 month, at least 3 months, at least 6 months, at least 1 year, at least 2 years, at least 3 years)].

2. A composition comprising:
a liposome-based nanocarrier comprising:
(i) a lipid;
(ii) an organic polymer; and
(iii) gamma-tocotrienol (GT3) (e.g., and/or an equivalent or derivative of GT3),
wherein the liposome-based nanocarrier has a surface having a negative charge.

3. The composition of claim 2, wherein the liposome-based nanocarrier further comprises a member (e.g., said member encapsulated inside of the liposome-based nanocarrier, incorporated into layers of the lipid, or covalently and/or non-covalently attached to the lipid on the surface of liposome-based nanocarrier) selected from the group consisting of:
a radioprotectant or radiation mitigator (e.g., Amifostine, N-acetylcystein, alpha-tocotrienol, gamma-tocotrienol, delta-tocotrienol, Genistein, rapamycin);
a growth factor (e.g., erythropoietin, granulocyte colony-stimulating factor, RNAi therapeutics (e.g., GTI-2040 (ribonucleotide reductase), SPC2996 (Bcl-2), LY2181308 (survivin), e.g., immunosuppressors (e.g., Tacrolimus, mTOR inhibitors, corticosteroids, antibiotics, epinephrine analogs, RNAi against Bim and PUMA));
a bisphosphonate (e.g., alendronate, ibandronate, risedronic acid, zoledonic acid);
a selective estrogen receptor modulator (e.g., raloxifene);
a parathyroid hormone modulator (e.g., teriparatide);
a biological (e.g., denosumab);
a chemotherapeutic drug (e.g., any of the chemotherapeutic drugs listed in Table 1);
a CXCR2 agonist (e.g., GROb, GROa) (e.g., for mobilizing stem cells);
a CXCR4 antagonist (e.g., AMD3100, BIO8020) (e.g., for mobilizing stem cells);
a HMG-CoA reductase (also known as 3-hydroxy-3-methyl-glutaryl-coenzyme A reductase or HMGCR) inhibitor (e.g., a statin (e.g. simvastatin, atorvastatin, lovastatin, pitavastatin)) for protecting marrow cells;
a superoxide dismutase (SOD) mimetic or other mimetics (e.g., manganese(III) 5,10,15,20-tetrakis(4-benzoic acid)porphyrin (MnTBAP), manganese (III) mesotetrakis (N-ethylpyridinium-2-yl) porphyrin (MnTE-2-PyP(5)) (AOEL 10113)) (e.g., for removing free radicals);
an androstenediol (e.g., for stimulating white blood cells (WBCs) and platelet growth); and
an androgen receptor (AR) agonist (e.g., dihydrotestosterone (DHT), a nonsteroidal selective androgen receptor (SARM) (e.g., BMS-564,929, VK5211) (e.g., for stimulating production of erythropoietin in the treatment of myeloid metaplasia).

4. The composition of claim 2 or 3, wherein the nanocarrier has up to 30 mol % GT3 (e.g., up to 24 mol % GT3, e.g., up to 10 mol % GT3) of the total moles comprising the nanocarrier.

5. The composition of any one of claims 2 to 4, wherein the lipid comprises one or more members selected from the group consisting of: cholesterol, 1,2-dioleoyl-sn-glycero-3-phosphoethanolamine-N-(succinyl) (succinyl-PE), 1,2-distearoyl-sn-glycero-3-phosphocholine (DSPC), cholesterol, 1,2-distearoyl-sn-glycero-3-phosphoethanolamine (DSPE), 1,2-dipalmitoyl-sn-glycero-3-phosphoethanolamine-N-(succinyl) (succinyl-DPPE), and 1,2-distearoyl-sn-glycero-3-phosphoethanolamine-N-[methoxy(polyethylene glycol)] (mPEG-DSPE).

6. The composition of any one of claims 2 to 5, wherein mPEG-DSPE is 1,2-distearoyl-sn-glycero-3-phosphoethanolamine-N-[methoxy(polyethylene glycol)-2000] (mPEG2000-DSPE).

7. The composition of any one of claims 2 to 6, wherein the lipid comprises cholesterol, DSPC, mPEG-DSPE, and succinyl-DPPE.

8. The composition of any one of claims 2 to 6, wherein the lipid comprises cholesterol, DSPC, mPEG-DSPE, and succinyl-PE.

9. The composition of any one of claims 2 to 8, wherein the lipid is labeled with an isotope.

10. The composition of any one claims 2 to 9, wherein the isotope is labeled through binding to a chelator.

11. The composition of any one of claims 2 to 10, wherein the isotope comprises a member selected from the group consisting of ³H, ⁶⁴Cu, ⁶⁶Ga, ⁸⁶Y, ¹¹¹In, ⁶⁷Ga, ⁶⁸Ga, ⁸⁹Zr, ^{124/131}I, and ¹⁷⁷Lu.

12. The composition of any one of claims 2 to 11, wherein the isotope comprises ³H.

13. The composition of any one of claims 2 to 11, wherein the isotope comprises ⁶⁴Cu.

14. The composition of claim of any one of claims 2 to 13, wherein the chelator comprises a member selected from the group consisting of 1,4,7,10-tetraazacyclododecane-1,4,7,10-tetraacetic acid (DOTA), 1,4,7-triazacyclononane-1,4,7-trisacetic acid (NOTA), and 1-(4-isothiocyanatophenyl)-3-[6,17-dihydroxy-7,10,18,21-tetraoxo-27-(N-acetylhydroxylamino)-6,11,17,22-tetraazaheptaeicosine] thiourea (DFO).

15. The composition of any one of claims 2 to 14, wherein the chelator comprises a member selected from the group consisting of DOTA-Bn-DSPE, NOTA-Bn-DSPE, and DFO-Bz-DSPE.

16. The composition of claim of any one of claims 2 to 15, wherein the organic polymer comprises polyethylene glycol (PEG).

17. The composition of any one of claims 2 to 16, wherein the nanocarrier comprises 1,2-distearoyl-sn-glycero-3-phosphoethanolamine-N-methoxy(polyethylene glycol) (mPEG-DSPE).

18. The composition of claim 17, wherein the concentration of mPEG-DSPE is from about 0.5 mole % to about 10 mole % of the total moles of the lipid comprising the nanocarrier.

19. The composition of any one of claim 17 or 18, wherein the concentration of mPEG-DSPE is from 0.5 mol % to about 1.5 mol % of the total moles of the lipid comprising the nanocarrier.

20. The composition of any one of claims 17 to 19, wherein the concentration of mPEG-DSPE is about 1 mole % of the total moles of the lipid comprising the nanocarrier.

21. The composition of any one of claims 16 to 20, wherein the concentration of succinyl-DPPE is from 5 mole % to 15 mole % (e.g., about 7 mole % to about 12 mole %, about 10 mole %) of the total moles of the lipid comprising the nanocarrier.

22. The composition of claim of any one of claims 2 to 21, wherein the liposome-based nanocarrier is at least 70 mol % lipid (e.g., at least 80 mole % lipid, at least 90 mole % lipid, at least 98 mole % lipid).

23. The composition of any one of claims 2 to 22, wherein the liposome-based nanocarrier has an average diameter in a range from 30 nm to 300 nm.

24. The composition of any one of claims 2 to 22, wherein the liposome-based nanocarrier has an average diameter in a range from about 70 nm to about 110 nm.

25. The composition of any one of claims 2 to 22, wherein the liposome-based nanocarrier has an average diameter of about 90 nm.

26. The composition of any one of claims 2 to 25, wherein the concentration of DPSC is from about 50 mole %-70 mole % (e.g., 55 mole %-75 mole %, e.g., about 60 mole %) of the total moles of the lipid comprising the nanocarrier.

27. The composition of any one of claims 2 to 26, wherein the concentration of cholesterol is from about 25 mole %-45 mole % (e.g., about 25 mole %-35 mole %, e.g., about 40 mole %) of the total moles of the lipid comprising the nanocarrier.

28. The composition of any one of claims 2 to 27, wherein the concentration of succinyl-PE is about 5 mole % to 15 mole % (e.g., about 7 mole % to 13 mole %, e.g., about 10 mole %) of the total moles of the lipid comprising the nanocarrier.

29. The composition of any one of claims 2 to 28, having one or more of (i), (ii), and (iii), as follows:
(i) from 3 to 20 mole % succinyl-DPPE;
(ii) from 0.5 to 2 mole % mPEG-DSPE; and
(iii) from 5 to 26 mole % an associated drug.

30. The composition of any one of claims 2 to 29, wherein the negative charge of the surface of the liposome-based nanocarrier as measured via the zeta potential at a pH of about 7.4 has a magnitude from about 5 mV to 25 mV (e.g., about 10 mV to 20 mV).

31. The composition of any one of claims 2 to 30, wherein a radiolabeling efficiency of the liposome-based nanocarrier is greater than 60% (e.g., greater than 70%) over at least a period of time after preparation.

32. The composition of any one of claims 2 to 31, wherein the diameter of the liposome varies no more than 30% (e.g., no more than 20%) over the period of time after preparation.

33. The composition of any one of claims 2 to 32, wherein the zeta potential as measured at about a pH 7.4 varies over a magnitude of no more than 5 mV (e.g., no more than 2.5 mV).

34. The composition of any one of claims 31 to 33, wherein the period of time is at least 2 weeks (e.g., at least 1 month, e.g., at least 3 months, e.g., at least 6 months, e.g., at least 12 months, e.g., at least 24 months).

35. A method for imaging a subject the method comprising:
administering to the subject a composition of any one of claims 2 to 34, wherein the lipid is labeled with an isotope.

36. The method of claim 35, further comprising obtaining and displaying a positron emission tomography (PET), single-photon emission computed tomography (SPECT), and/or Positron emission tomography-computed tomography (PET/CT) image of at least one tissue of the subject comprising the composition.

37. The method of claim 35 or 36, further comprising quantitatively measuring a distribution of the composition in at least one tissue of the subject.

38. The method of claim 37, the method comprising
quantitatively measuring the distribution of the composition in an organ of the reticuloendothelial system.

39. The method of claim 38, wherein the organ comprises a member selected from the group consisting of: liver, spleen, and bone marrow.

40. The method of claim 39, wherein the organ comprises a member selected from the group consisting of: spleen and bone marrow.

41. The method of any one of claims 36 to 38, comprising determining a concentration and/or total amount of delivered radiolabeled drug in the tissue based on a positron emission tomography (PET), single-photon emission computed tomography (SPECT), or Positron Emission Tomography-Computed Tomography (PET/CT) image of the tissue.

42. The method of any one of claims 37 to 41, the method comprising
quantitatively measuring the distribution of the composition in one or more lymph nodes.

43. The method of any one of claims 37 to 41, the method comprising quantitatively measuring the distribution of the composition in bone marrow.

44. The method of any one of claims 37 to 41, the method comprising quantitatively measuring the distribution of the composition in spleen.

45. The method of any one of claims 35 to 44, wherein the administered composition demonstrates selective targeting of bone marrow of the subject such that concentration of the composition in bone marrow is at least 3 fold greater than the concentration of the composition in any of the tumor tissue at a given time following administration of the composition, wherein the given time is at least 1 hour following administration.

46. The method of any one of claims 35 to 45, further comprising capturing and displaying a sequence of PET images in real time.

47. A method of treating a subject, the method comprising administering the composition of any one of claims 2 to 34 to the subject suffering from or susceptible to a disease and/or condition.

48. The method of claim 47, wherein the disease and/or condition comprises a member selected from the group consisting of bone marrow suppression (BMS), myelodysplastic syndrome (MDS), acute myeloid leukemia (AML), chronic myeloid leukemia (CML), sepsis, graft-versus-host-disease (GVHD), bone metastasis, osteoporosis, and myeloid metaplasia.

49. The method of claim 47 or 48, wherein the disease and/or condition comprises exposure to radiation.

50. The method of claim 48, the method further comprising
after administering the composition, administering a chemotherapeutic and/or radiation therapy.

51. The method of any one of claims 48 to 50, the method further comprising before administering the composition at one or more time points (e.g., six time points), administering a chemotherapeutic and/or radiation therapy.

52. The method of any one of claims 48 to 51, wherein the composition is administered at the dosage from about 15 to about 50 mg/kg.

53. The method of any one of claims 48 to 51, wherein the composition is administered at the dosage of about 32 mg/kg

54. The method of any one of claims 48 to 51, wherein the composition is administered at the dosage of about 40 mg/kg.

55. The method of any one of claims 47 to 54, wherein the administered composition demonstrates selective targeting of bone marrow of the subject such that the concentration of the liposome-based nanocarrier in bone marrow is at least 3 fold greater than the concentration of the liposome-based nanocarrier in any of the tumor tissue at a given time following administration of composition, wherein the given time is at least 1 hour, following administration.

56. The method of any one of claims 47 to 55, wherein the administered composition protects bone marrow cells against radiation (e.g., wherein the subject has received or will receive radiation exposure, e.g., from a chemotherapy drug, radiation treatment, and/or other radiation exposure).

57. The method of any one of claims 47 to 56, wherein the administered composition mobilizes and recruits stem cells to the bone marrow (e.g., wherein the subject has received or will receive radiation exposure, e.g., from a chemotherapy drug, radiation treatment, and/or other radiation exposure).

58. The method of any one of claims 47 to 57, wherein the administered composition stimulates production of erythropoietin (e.g., wherein the subject has received or will receive radiation exposure, e.g., from a chemotherapy drug, radiation treatment, and/or other radiation exposure).

59. A method of monitoring a patient, the method comprising:
administering the composition of any one of claims 2 to 34 to a patient suffering from or susceptible to a disease and/or condition; and
investigating a quantity of drug (e.g., a drug currently or having been associated with the liposome-based nanocarrier of the composition) delivered to at least one tissue of the patient.

60. A method of imaging an organ of the reticuloendothelial system in a subject, the method comprising:
detecting radiation from the liposome-based nanocarrier of any one of claims 2 to 34, the subject having been administered the composition.

61. The method of claim 60, wherein the radiation is detected via an external PET imaging system.

62. The method of claim 60 or 61, wherein the organ comprises a member selected from the group consisting of active bone marrow, liver, and spleen.

63. The method of any one of claims 60 to 62, wherein the organ comprises a member selected from the group consisting of active bone marrow and spleen.

64. The method of any one of claims 60 to 63, the method further comprising displaying an image corresponding to the detected radiation, the image visually distinguishing active bone marrow from other tissue and, optionally, quantifying the concentration of drug and/or liposome-based nanocarrier.

65. A composition comprising:
a liposome-based nanocarrier comprising:
(i) a lipid;
(ii) an organic polymer; and
(iii) gamma-tocotrienol (GT3) (e.g., and/or an equivalent or derivative of GT3), and
an associated drug (e.g., wherein the associated drug is encapsulated inside of the liposome-based nanocarrier, incorporated into layers of the lipid, or covalently and/or non-covalently attached to the lipid on the surface of liposome-based nanocarrier),
for use in a method of treating a disease and/or condition in a subject, wherein the treating comprises delivering the composition to the subject.

66. A composition comprising:
a liposome-based nanocarrier comprising:
(i) a lipid;
(ii) an organic polymer; and
(iii) gamma-tocotrienol (GT3) (e.g., and/or an equivalent or derivative of GT3),
wherein the liposome-based nanocarrier has a surface having a negative charge, and
an associated drug (e.g., wherein the associated drug is encapsulated inside of the liposome-based nanocarrier, incorporated into layers of the lipid, or covalently and/or non-covalently attached to the lipid on the surface of liposome-based nanocarrier),
for use in a method of monitoring of a disease and/or condition in a subject, wherein the monitoring comprises delivering the composition to the subject.

67. A composition comprising:
a liposome-based nanocarrier comprising:
(i) a lipid;
(ii) an organic polymer; and
(iii) gamma-tocotrienol (GT3) (e.g., and/or an equivalent or derivative of GT3),
wherein the liposome-based nanocarrier has a surface having a negative charge, and
an associated drug (e.g., wherein the associated drug is encapsulated inside of the liposome-based nanocarrier, incorporated into layers of the lipid, or covalently and/or non-covalently attached to the lipid on the surface of liposome-based nanocarrier),
for use in (a) a method of treating a disease and/or condition in a subject or (b) in a method of monitoring of a disease and/or condition in a subject, wherein the monitoring comprises delivering the composition to the subject.

68. A composition comprising:
a liposome-based nanocarrier comprising:
(i) a lipid;
(ii) an organic polymer; and
(iii) gamma-tocotrienol (GT3) (e.g., and/or an equivalent or derivative of GT3),
wherein the liposome-based nanocarrier has a surface having a negative charge, and
an associated drug (e.g., wherein the associated drug is encapsulated inside of the liposome-based nanocarrier, incorporated into layers of the lipid, or covalently and/or non-covalently attached to the lipid on the surface of liposome-based nanocarrier),
for use in therapy.

69. A kit (e.g., for use in radioprotection or radiation mitigation, e.g., for use in methods of treating, monitoring, and/or imaging a subject and/or patient) comprising:
a first container (e.g., an ampule, a vial, a cartridge, a reservoir, a lyo-ject, or a pre-filled syringe), wherein the first container contains a composition of any one of claims 2 to 34 or 64 to 68.

70. The kit of claim 69, comprising a neutralizing solution, wherein the neutralizing solution is contained in a second container (e.g., wherein the neutralizing solution adjusts the pH to about pH 7.0-7.4) (e.g., wherein the neutralizing solution comprises potassium carbonate, e.g., 500 mM potassium carbonate).

71. The kit of claim 70, wherein the neutralizing solution comprises potassium carbonate.

72. The kit of claim any one of claims 69 to 71, comprising a Solution A, wherein the Solution A is contained in a third container. (e.g., wherein the Solution A enhances labeling efficiency, e.g., wherein Solution A reduces pH) (e.g., wherein the Solution A comprises ammonium acetate, e.g., 500 mM ammonium acetate).

73. The kit of claim 72, wherein the Solution A comprises ammonium acetate.

74. The kit of any one of claims 69 to 73, comprising a radiolabel (e.g., ⁶⁴Cu, e.g., ⁶⁴Cu—Cl₂, e.g., ⁸⁹Zr), wherein the radiolabel is contained in a fourth container.

75. A method for preparing a composition (e.g., a composition of any one of claims 1 to 32 or 63 to 67), the method comprising:
contacting (e.g., via gentle swirling and/or mixing) a first solution with a substance comprising a radiolabel (e.g., ⁶⁴Cu, e.g., ⁶⁴Cu—Cl₂, e.g., ⁸⁹Zr) to generate a second solution (e.g., wherein the average labeling yield is greater than 70%, greater than 80%, greater than 90%),
wherein the first solution comprises a liposome-based nanocarrier comprising:
(i) a lipid;
(ii) an organic polymer; and
(iii) gamma-tocotrienol (GT3) (e.g., and/or an equivalent or derivative of GT3),
wherein the liposome-based nanocarrier has a surface having a negative charge;
heating (e.g., at 50° C.) the second solution for a period of time (e.g., for about 10 minutes, for about 20 minutes, for about 30 minutes, for about 40 minutes); and
contacting (e.g., aseptically) (e.g., via gentle swirling and/or mixing) the second solution with a Neutralizing Solution to generate a third solution (e.g., thereby adjusting pH of the third solution to a value of about 7.0 to about 7.4) (e.g., wherein the third solution comprises the composition of any one of claims 1 to 14 or 37 to 41) (e.g., wherein the third solution has a pH value of from about 7.0 to about 7.4).

76. The method of claim 75, the method further comprising:
contacting (e.g., aseptically) (e.g., via gentle swirling and/or mixing) a Solution A with the liposome-based nanocarrier, thereby generating the first solution [e.g., enhances efficiency particularly where radiolabel comprises ⁶⁴Cu] wherein the Solution A enhances radiolabeling efficiency, e.g., wherein Solution A reduces pH of the first solution (e.g., wherein the Solution A comprises ammonium acetate, e.g., 500 mM ammonium acetate)].

77. The method of claim 75 or 76, wherein the Neutralizing Solution comprises potassium carbonate, e.g., 500 mM potassium carbonate.

78. A method for using the kit of any one of claims 69 to 74, the method comprising:
receiving the kit (e.g., via mail, e.g., via courier);
storing the kit (e.g., storing the kit at about 4° C.) for a storage duration; and
administering the kit to a subject after the storage duration [e.g., wherein the storage duration is at least 2 weeks (e.g., at least 1 month, at least 3 months, at least 6 months, at least 1 year, at least 2 years, at least 3 years)].

79. The method of claim 78, wherein the method comprises transporting the kit (e.g., sending via mail, e.g., sending via courier) at a temperature of about 4° C. (e.g., about 2° C., about 5° C., about 10° C.).

80. A composition comprising:
a liposome-based nanocarrier comprising:
(i) a lipid;
(ii) an organic polymer;
and wherein the liposome-based nanocarrier has a surface having a negative charge, and
an associated drug (e.g., wherein the associated drug is encapsulated inside of the liposome-based nanocarrier, incorporated into layers of the lipid, or covalently and/or non-covalently attached to the lipid on the surface of liposome-based nanocarrier).

81. The composition of claim 80, wherein the associated drug comprises a member selected from the group consisting of:
a free radical scavenger (e.g., gamma-tocotrienol (GT3));
a radioprotectant or radiation mitigator (e.g., Amifostine, N-acetylcystein, alpha-tocotrienol, gamma-tocotrienol, delta-tocotrienol, Genistein, rapamycin);
a growth factor (e.g., erythropoietin, granulocyte colony-stimulating factor, RNAi therapeutics (e.g., GTI-2040 (ribonucleotide reductase), SPC2996 (Bcl-2), LY2181308 (survivin), e.g., immunosuppressors (e.g., Tacrolimus, mTOR inhibitors, corticosteroids, antibiotics, epinephrine analogs, RNAi against Bim and PUMA));
a bisphosphonate (e.g., alendronate, ibandronate, risedronic acid, zoledonic acid);
a selective estrogen receptor modulator (e.g., raloxifene);
a parathyroid hormone modulator (e.g., teriparatide);
a biological (e.g., denosumab);
a chemotherapeutic drug (e.g., any of the chemotherapeutic drugs listed in Table 1);
a CXCR2 agonist (e.g., GROb, GROa) (e.g., for mobilizing stem cells);
a CXCR4 antagonist (e.g., AMD3100, BIO8020) (e.g., for mobilizing stem cells);
a HMG-CoA reductase (also known as 3-hydroxy-3-methyl-glutaryl-coenzyme A reductase or HMGCR) inhibitor (e.g., a statin (e.g. simvastatin, atorvastatin, lovastatin, pitavastatin)) for protecting marrow cells;
a superoxide dismutase (SOD) mimetic or other mimetics (e.g., manganese(III) 5,10,15,20-tetrakis(4-benzoic acid)porphyrin (MnTBAP), manganese (III) mesotetrakis (N-ethylpyridinium-2-yl) porphyrin (MnTE-2-PyP(5+)) (AOEL 10113)) (e.g., for removing free radicals);
an androstenediol (e.g., for stimulating white blood cells (WBCs) and platelet growth); and
an androgen receptor (AR) agonist (e.g., dihydrotestosterone (DHT), a nonsteroidal selective androgen receptor (SARM) (e.g., BMS-564,929, VK5211) (e.g., for stimulating production of erythropoietin in the treatment of myeloid metaplasia).

82. The composition of claim 80 or 81, wherein the nanocarrier has up to 30 mole % GT3 (e.g., up to 24 mole % GT3, e.g., up to 10 mole % GT3) of the total moles comprising the nanocarrier.

83. The composition of any one of claims 80 to 82, wherein the lipid comprises one or more members selected from the group consisting of: cholesterol; 1,2-dioleoyl-sn-glycero-3-phosphoethanolamine-N-(succinyl) (succinyl-PE); 1,2-distearoyl-sn-glycero-3-phosphocholine (DSPC); 1,2-distearoyl-sn-glycero-3-phosphoethanolamine (DSPE); 1,2-dipalmitoyl-sn-glycero-3-phosphoethanolamine-N-(succinyl) (succinyl-DPPE); and 1,2-distearoyl-sn-glycero-3-phosphoethanolamine-N-[methoxy(polyethylene glycol)] (mPEG-DSPE).

84. The composition of any one of claims 80 to 83, wherein mPEG-DSPE is 1,2-distearoyl-sn-glycero-3-phosphoethanolamine-N-[methoxy(polyethylene glycol)-2000] (mPEG2000-DSPE).

85. The composition of any one of claims 80 to 84, wherein the lipid comprises cholesterol, DSPC, mPEG-DSPE, and succinyl-PE.

86. The composition of any one of claims 80 to 85, wherein the lipid is labeled with an isotope.

87. The composition of any one of claims 80 to 86, wherein the isotope is labeled through binding to a chelator.

88. The composition of any one of claims 80 to 87, wherein the isotope comprises a member selected from the group consisting of ³H, ⁶⁴Cu, ⁶⁶Ga, ⁸⁶Y, ¹¹¹In, ⁶⁷Ga, ⁶⁸Ga, ⁸⁹Zr, ^{124/131}I, and ¹⁷⁷Lu.

89. The composition of any one of claims 80 to 88, wherein the isotope comprises ³H.

90. The composition of any one of claims 80 to 89, wherein the isotope comprises ⁶⁴Cu.

91. The composition of any one of claims 80 to 90, wherein the chelator comprises a member selected from the group consisting of 1,4,7,10-tetraazacyclododecane-1,4,7,10-tetraacetic acid (DOTA), 1,4,7-triazacyclononane-1,4,7-trisacetic acid (NOTA), and 1-(4-isothiocyanatophenyl)-3-[6,17-dihydroxy-7,10,18,21-tetraoxo-27-(N-acetylhydroxylamino)-6,11,17,22-tetraazaheptaeicosine] thiourea (DFO).

92. The composition of any one of claims 80 to 91, wherein the chelator comprises a member selected from the group consisting of DOTA-Bn-DSPE, NOTA-Bn-DSPE, and DFO-Bz-DSPE.

93. The composition of claim of any one of claims 80 to 92, wherein the organic polymer comprises polyethylene glycol (PEG).

94. The composition of any one of claims 80 to 93, wherein the nanocarrier comprises 1,2-distearoyl-sn-glycero-3-phosphoethanolamine-N-methoxy(polyethylene glycol) (mPEG-DSPE).

95. The composition of claim 94, wherein the concentration of mPEG-DSPE is from about 0.5 mole % to about 10 mole % of the total moles of the lipid comprising the nanocarrier.

96. The composition of any one of claim 94 or 95, wherein the concentration of mPEG-DSPE is from 0.5 mole % to about 1.5 mole % of the total moles of the lipid comprising the nanocarrier.

97. The composition of any one of claims 94 to 96, wherein the concentration of mPEG-DSPE is about 1 mole % of the total moles of the lipid comprising the nanocarrier.

98. The composition of any one of claims 94 to 97, wherein the concentration of succinyl-DPPE is from 5 mole % to 15 mole % (e.g., about 7 mole % to about 12 mole %, about 10 mole %) of the total moles of the lipid comprising the nanocarrier.

99. The composition of claim of any one of claims 80 to 98, wherein the liposome-based nanocarrier is at least 70 mole % lipid (e.g., at least 80 mole % lipid, at least 90 mole % lipid, at least 98 mole % lipid).

100. The composition of any one of claims 80 to 99, wherein the liposome-based nanocarrier has an average diameter in a range from 30 nm to 300 nm.

101. The composition of any one of claims 80 to 100, wherein the liposome-based nanocarrier has an average diameter in a range from about 70 nm to about 110 nm.

102. The composition of any one of claims 80 to 101, wherein the liposome-based nanocarrier has an average diameter of about 90 nm.

103. The composition of any one of claims 80 to 102, wherein the concentration of DPSC is from about 50 mole %-70 mole % (e.g., 55 mole %-75 mole %, e.g., about 60 mole %) of the total moles of the lipid comprising the nanocarrier.

104. The composition of any one of claims 80 to 103, wherein the concentration of cholesterol is from about 25 mole %-45 mole % l (e.g., about 25 mole % l-35 mole %, e.g., about 40 mole %) of the total moles of the lipid comprising the nanocarrier.

105. The composition of any one of claims 80 to 104, wherein the concentration of succinyl-PE is about 5 mole % to 15 mole % (e.g., about 7 mole % to 13 mole %, e.g., about 10 mole %) of the total moles of the lipid comprising the nanocarrier.

106. The composition of any one of claims 80 to 105, wherein the negative charge of the surface of the liposome-based nanocarrier as measured via the zeta potential at a pH of about 7.4 has a magnitude from about 5 mV to 25 mV (e.g., about 10 mV to 20 mV).

107. The composition of any one of claims 80 to 106, wherein a radiolabeling efficiency of the liposome-based nanocarrier is greater than 60% (e.g., greater than 70%) over at least a period of time after preparation.

108. The composition of any one of any one of claims 80 to 107, wherein the diameter of the liposome varies no more than 30% (e.g., no more than 20%) over the period of time after preparation.

109. The composition of any one of claims 80 to 108, wherein the zeta potential as measured at about a pH 7.4 varies over a magnitude of no more than 5 mV (e.g., no more than 2.5 mV).

110. The composition of any one of claims 107 to 109, wherein the period of time is at least 2 weeks (e.g., at least 1 month, e.g., at least 3 months, e.g., at least 6 months, e.g., at least 12 months, e.g., at least 24 months).
